SEQUENCE LISTING (22 sequences)

SEQ ID NO: 1 - protein, 87 aa, Unknown
  Description of Unknown Human or mouse polypeptide
NYFTKRGYAWDGKSVVPEKPKKVKDPLIKKNNTYAPTATSYTPNLARGDPGLATIAKSATIEPKEVKPETKPPEPKKTFNSVSKIDR

SEQ ID NO: 2 - protein, 179 aa, Homo sapiens
NYFTNIQMEKAKRKTSKPPQEVPAAPVQREKHPEAPLQNTNANLNMRKRTNALVSESDVGNRTEVGNHSSKSSTVVQESSKGTPRSYLASSPNPFSRANAAETISAARALPSASPTSIRTGYMPRKASVGSASTRHVFGSRLQRIKTTVNTIGATGKLSATPPPSAPPPSGSGTSKIDK

SEQ ID NO: 3 - protein, 178 aa, Mus musculus
NYFTNIQMQKAKKKISKPPPEVPAAPVLKEKHTETSLQNTHANLNMRKRTNALVHSESDVKSRTEVGNHSSKTSAVQESSEATPKAHLASSPNPFSRANAAETMSAAARGLSSAASPSPHGTLRPASLGSASTRPAFGSRLGRIKTTVNTTGAAGNVSATPPPPAPPPSGSGTSKIDK

SEQ ID NO: 4 - protein, 38 aa, Artificial Sequence
  Description of Artificial Sequence Synthetic polypeptide
KKKISKPPPEVPAAPVLKEKHTETSLQNTHANLNMRKR

SEQ ID NO: 5 - protein, 38 aa, Artificial Sequence
  Description of Artificial Sequence Synthetic polypeptide
QQQISKPPPEVPAAPVLKEKHTETSLQNTHANLNMRKR

SEQ ID NO: 6 - protein, 38 aa, Artificial Sequence
  Description of Artificial Sequence Synthetic polypeptide
KKKISKPPPEVPAAPVLKEKHTETSLQNTHANLNMQQQ

SEQ ID NO: 7 - protein, 38 aa, Artificial Sequence
  Description of Artificial Sequence Synthetic polypeptide
KKKISKPPPEVPAAPVLKEKHTETSLQNTHANLNMQKR

SEQ ID NO: 8 - protein, 38 aa, Artificial Sequence
  Description of Artificial Sequence Synthetic polypeptide
KKKISKPPPEVPAAPVLKEKHTETSLQNTHANLNMRQR

SEQ ID NO: 9 - protein, 38 aa, Artificial Sequence
  Description of Artificial Sequence Synthetic polypeptide
KKKISKPPPEVPAAPVLKEKHTETSLQNTHANLNMRKQ

SEQ ID NO: 10 - protein, 38 aa, Artificial Sequence
  Description of Artificial Sequence Synthetic polypeptide
KKKISKPPPEVPAAPVLKEKHTETSLQNTHANLNMRKK

SEQ ID NO: 11 - DNA, 1975 bp, Mus musculus
  CDS (1)..(1656)
  CDS (1660)..(1710)
  CDS (1714)..(1746)
  CDS (1750)..(1803)
  CDS (1807)..(1854)
  CDS (1864)..(1890)
  CDS (1894)..(1929)
  CDS (1939)..(1974)
atggtttctgtccagaaggtacccgcgattgcgctgtgctccggggtcagcctcgccctcctgcacttcctgtgcctggcggcttgtttaaacgaatccccaggacagaactcaaaggacgagaaattgtgcccggaaaattttacccgtattctggacagtttgctggatggttatgacaacaggctgcgtcctggatttgggggtcctgttacagaagtgaaaactgatatatatgtcaccagctttgggcccgtttctgatgttgaaatggaatacactatggatgtgttcttcagacagacatggattgacaaaagactaaaatatgacggcccaattgaaatcttgaggctgaataatatgatggtcaccaaagtttggacccctgatactttcttcaggaatggaaagaaatctgtctcacataacatgacagctccaaataagctttttagaattatgagaaatggcactattttatacacaatgagactcaccataagtgcggagtgccccatgagactggtggattttcctatggatggtcatgcctgccctttgaaatttgggagttatgcatatcccaaaagtgagatgatctacacctggaccaaaggccctgagaagtcagtggaggtgccaaaggagtcttctagcttagttcaatatgacctcattgggcagactgtatcaagcgagactatcaaatctattacaggtgaatacattgttatgacggtttacttccacctcagacggaagatgggctactttatgattcagacgtatatcccatgcatcatgacagtgattctttctcaagtttccttctggataaacaaggagtctgttccagctagaactgtatttggaataaccacagtcctcacgatgaccaccctaagcatcagtgctcggcattctttgcccaaagtgtcctatgcgactgccatggattggttcatagctgtctgttttgcttttgtattttcggctcttattgagtttgctgctgtcaactatttcaccaacattcaaatgcagaaagccaaaaagaagatatcaaagcctcccccagaagttccagctgctcctgtgctgaaggagaaacacacagaaacatcccttcagaatacacatgccaatttgaacatgaggaaaagaacaaatgccttggtccattcagaatcggatgtcaaaagcagaactgaggtgggaaatcactccagcaagaccagcgctgtccaggagtcttctgaagccacgcctaaggctcacttagcttccagtccaaatccattcagcagggcaaatgcagctgagactatgtctgctgcagccagaggtctttcatctgcagcatccccctctccgcatggcacattgcggccagcttctttggggtcagcttccactcgccctgcatttggatctagacttgggcgaattaagacaacagttaatacaacaggggctgctgggaatgtgtcagccacacctcctccccctgctccaccgccttctggatctggcacaagtaaaatagacaaatatgctcgtattctctttccagtcacatttggagcatttaacatggtctactgggttgtttatttatctaaggacaccatggagaaatcagaaagtctaatgtaattttgttgctatagtaatttcataacaacgatggaaatacagactgtcttttaaaatgtttttaaaggtaagtattctttactaaaataaaaattctatgtaattttttcacttaaaaaaatacaagtcagatatggagagagttaattaactcttcagtggctagaaagtgaaccatctttctttatagaaagatagctttaaaagaatctattcagtgttcaaattggatagaacacagagcattatggaaagttggagcaagagaaatagagctaggagagatatgtggtacacccttttgcattgaaatttg

SEQ ID NO: 12 - protein, 552 aa, Mus musculus
MVSVQKVPAIALCSGVSLALLHFLCLAACLNESPGQNSKDEKLCPENFTRILDSLLDGYDNRLRPGFGGPVTEVKTDIYVTSFGPVSDVEMEYTMDVFFRQTWIDKRLKYDGPIEILRLNNMMVTKVWTPDTFFRNGKKSVSHNMTAPNKLFRIMRNGTILYTMRLTISAECPMRLVDFPMDGHACPLKFGSYAYPKSEMIYTWTKGPEKSVEVPKESSSLVQYDLIGQTVSSETIKSITGEYIVMTVYFHLRRKMGYFMIQTYIPCIMTVILSQVSFWINKESVPARTVFGITTVLTMTTLSISARHSLPKVSYATAMDWFIAVCFAFVFSALIEFAAVNYFTNIQMQKAKKKISKPPPEVPAAPVLKEKHTETSLQNTHANLNMRKRTNALVHSESDVKSRTEVGNHSSKTSAVQESSEATPKAHLASSPNPFSRANAAETMSAAARGLSSAASPSPHGTLRPASLGSASTRPAFGSRLGRIKTTVNTTGAAGNVSATPPPPAPPPSGSGTSKIDKYARILFPVTFGAFNMVYWVVYLSKDTMEKSESLM

SEQ ID NO: 13 - protein, 17 aa, Mus musculus
FCCYSNFITTMEIQTVF

SEQ ID NO: 14 - protein, 11 aa, Mus musculus
NVFKGKYSLLK

SEQ ID NO: 15 - protein, 18 aa, Mus musculus
KFYVIFSLKKIQVRYGES

SEQ ID NO: 16 - protein, 16 aa, Mus musculus
LTLQWLESEPSFFIER

SEQ ID NO: 17 - protein, 9 aa, Mus musculus
KNLFSVQIG

SEQ ID NO: 18 - protein, 12 aa, Mus musculus
NTEHYGKLEQEK

SEQ ID NO: 19 - protein, 12 aa, Mus musculus
ERYVVHPFALKF

SEQ ID NO: 20 - DNA, 1662 bp, Homo sapiens
  CDS (1)..(1662)
atggtttctgccaagaaggtacccgcgatcgctctgtccgccggggtcagtttcgccctcctgcgcttcctgtgcctggcggtttgtttaaacgaatccccaggacagaaccaaaaggaggagaaattgtgcacagaaaatttcacccgcatcctggacagtttgctcgatggttatgacaacaggctgcgtcctggatttgggggtcctgttacagaagtgaaaactgacatatatgtcaccagctttggacctgtttctgatgttgaaatggaatacacaatggatgtgttcttcaggcagacatggattgacaaaagattaaaatatgacggccccattgaaattttgagattgaacaatatgatggtaacgaaagtgtggacccctgatactttcttcaggaatggaaagaaatctgtctcacataatatgacagctccaaataagctttttagaattatgagaaatggtactattttatacacaatgagactcaccataagtgcggagtgtcccatgagattggtggattttcccatggatggtcatgcatgccctttgaaattcgggagttatgcctatccaaagagtgagatgatctatacctggacaaaaggtcctgagaaatcagttgaagttccgaaggagtcttccagcttagttcaatatgatttgattgggcaaaccgtatcaagtgaaaccatcaaatcaattacgggtgaatatattgttatgacggtttacttccacctcagacggaagatgggttattttatgattcagacctatattccgtgcattatgacagtgattctttctcaagtttcattttggataaataaagaatcagttcccgctaggactgtatttggaataacaactgtcctcaccatgaccacactaagcatcagtgcacgacattctttgcccaaagtgtcctatgctaccgccatggactggttcatagctgtctgctttgcttttgtattttcggcccttatcgagtttgctgctgtcaactatttcaccaatattcaaatggaaaaagccaaaaggaagacatcaaagccccctcaggaagttcccgctgctccagtgcagagagagaagcatcctgaagcccctctgcagaatacaaatgccaatttgaacatgagaaaaagaacaaatgctttggttcactctgaatctgatgttggcaacagaactgaggtgggaaaccattcaagcaaatcttccacagttgttcaagaatcttctaaaggcacacctcggtcttacttagcttccagtccaaacccattcagccgtgcaaatgcagctgaaaccatatctgcagcaagagcacttccatctgcttctcctacttctatccgaactggatatatgcctcgaaaggcttcagttggatctgcttctactcgtcacgtgtttggatcaagactgcagaggataaagaccacagttaataccataggggctactgggaagttgtcagctactcctcctccatcggctccaccaccttctggatctggcacaagtaaaatagacaaatatgcccgtattctctttccagtcacatttggggcatttaacatggtttattgggttgtttatttatctaaggacactatggagaaatcagaaagtctaatg

SEQ ID NO: 21 - protein, 554 aa, Homo sapiens [sequence omitted]

SEQ ID NO: 22 - DNA, 44 bp, Mus musculus
gccaatttgaacatgaggaaacaaacaaatgccttggtccattc

(19) United States
(12) Patent Application Publication
(10) Pub. No.: US 2009/0269795 A1
(43) Pub. Date: Oct. 29, 2009

(54) Mutant alpha4betadelta GABAA receptor and methods of treating anxiety or irritability

(75) Inventor: Sheryl Smith, Alantic Beach, NY (US)

Correspondence Address: DILWORTH & BARRESE, LLP, 1000 WOODBURY ROAD, SUITE 405, WOODBURY, NY, 11797, US

(73) Assignee: The Research Foundation of State University of New York, Albany, NY (US)

(21) Appl. No.: 12/075,159

(22) Filed: Mar. 10, 2008

Related U.S. Application Data
(60) Provisional application No. 60/906,165, filed on Mar. 9, 2007.

(51) Int. Cl. C12Q 1/02; A61K 31/437; C07K 14/705; C07H 21/00; A61K 31/711; C12N 15/63; C12N 5/10

(52) U.S. Cl. 435/29; 514/302; 530/350; 536/23.1; 514/44 R; 435/320.1; 435/325

(57) ABSTRACT

The present invention provides methods for treating anxiety or irritability in a subject. The methods comprise administering to the subject an effective amount of an antagonist of allopregnanolone (THP), or a regulator which decreases expression of the alpha 4 subunit of GABA such as gabadoxbol (THIP), or a vector comprising an isolated nucleic acid molecule encoding a mutant alpha 4 subunit GABA_{A} receptor protein having a neutral or non-basic amino acid residue substituted for the arginine residue at position 353 of the wild type mature protein, wherein this nucleic acid molecule is operably linked to a promoter which functions in the human brain. Such methods are useful in treating a subject undergoing a stage such as entering or having reached puberty, suffering from pre-menstrual syndrome (PMS), entering or having reached post-partem stage, entering or having reached menopause, and/or suffering from chronic stress. Also provided by the present isolated is a mutant alpha 4 subunit of GABA_{A} receptor protein which has a neutral or non-basic amino acid residue substituted for the arginine residue at position 353 of the wild type mature protein and an isolated nucleic acid molecule encoding this mutated protein. The present invention also provides vectors comprising a subject isolated nucleic acid molecule operably liked to a promoter which functions in prokaryotic or eukaryotic cells, as well as host cells comprising such vectors. In addition, the present invention provides methods for identifying an antagonist of THP.

### CROSS REFERENCE TO RELATED APPLICATIONS

This application claims priority from U.S. Provisional Application No. 60/906,165, filed Mar. 9, 2007, which is incorporated by its entirety herein.

### BACKGROUND OF THE INVENTION

The onset of puberty is associated with increases in emotional reactivity and anxiety^{1,2}. Responses to stressful events are amplified³, and anxiety and panic disorder first emerge at this time², being twice as likely to occur in girls than in boys². Few studies have addressed the biological basis of this important issue, although suicide risk increases in adolescence, despite the use of adult-based medical strategies².

A brain molecule known as the GABA_{A} receptor plays a pivotal role in the generation of anxiety⁴. This receptor is the target for endogenous steroids such as THP (3α-OH-5α[β]-pregnan-20-one or [allo]pregnanolone), which increase GABA-gated currents at physiological concentrations⁵ of the steroid. THP is a metabolite of the ovarian/adrenal steroid progesterone, but is also formed in the brain as a compensatory response to stress⁶. In adults, THP potently reduces anxiety in humans⁷, an effect seen in animal models with direct administration into the dorsal CA1 hippocampus⁸, part of the limbic system that regulates emotion. It is generally accepted that the GABA-enhancing action of THP underlies its well-known anxiety-reducing effect in adults, which is similar to other GABA-enhancing drugs such as the benzodiazepines.

GABA_{A} receptors are pentamers formed predominantly of 2α, 2β and 1γ subunits⁹ which gate a Cl⁻ current and produce most fast synaptic inhibition in the brain. Substitution of the δ subunit for γ2 yields a receptor with the highest sensitivity to steroids such as THP¹⁰⁻¹². These highly sensitive δ-GABA_{A} receptors are extrasynaptic¹³, and mediate tonic rather than synaptic inhibition in areas such as dentate gyrus¹⁴. Thus, THP and related steroids enhance inhibition here by selectively increasing the tonic current¹⁴ at physiological concentrations (<40 nM)¹⁵.

Expression of α4 βδ GABA_{A} receptors is normally very low in other areas of the brain, such as the CA1 hippocampus¹⁶, one area that regulates anxiety. However, fluctuating levels of THP can increase expression of α4 and δ subunits in this region, an effect tightly correlated with increased anxiety in adult female rodents¹⁷⁻¹⁹. Because the onset of puberty is a naturally occurring hormonal transition state associated with increases in anxiety, we tested whether pubertal development was associated with increased expression of these steroid-sensitive α4 βδ GABA_{A} receptors in CA1 hippocampus.

In addition to altered expression of α4 βδ receptors, other factors determine the level of inhibition in CNS circuits. In particular, the direction of Cl⁻ current varies across CNS regions: In limbic regions of the brain which normally express α4 βδ GABA_{A} receptors, such as the dentate gyrus, the Cl⁻ current is inward (i.e., outward Cl⁻ flux)²⁰. However, in CA1 hippocampal pyramidal cells, both dendritic and somatic GABAergic currents are normally outward in response to low concentrations of GABA²¹⁻²³, as would be found extrasynaptically²⁴. In work leading up to this invention, it also determined if the effect of THP on α4 β2δ receptors depended on the direction of Cl⁻ current using patch clamp recording techniques with recombinant receptors expressed in human embryonic kidney (HEK)-293 cells as well as in hippocampal slices. Other studies leading to the present invention were designed to determine whether the anxiety response to stress at puberty in females involves a change in the response of GABA_{A} receptors to a stress steroid.

### SUMMARY OF THE INVENTION

The present invention provides a method for treating anxiety or irritability in a subject. The method comprises administering to the subject an effective amount of an antagonist of allopregnanolone (THP). The method is useful of treating patients undergoing a stage such as entering or having reached puberty, suffering from pre-menstrual syndrome (PMS), entering or having reached post-partem stage, entering or having reached menopause, and/or suffering from chronic stress.

The present invention also provides a method for treating anxiety or irritability in a subject by administering to the subject an effective amount of a regulator which decreases expression of the alpha 4 subunit of GABA. Such method is useful in treating a subject undergoing a stage such as entering or having reached puberty, suffering from pre-menstrual syndrome (PMS), entering or having reached post-partem stage, entering or having reached menopause, and/or suffering from chronic stress. An example of a regulator useful in practicing this aspect of the invention is gabadoxbol (THIP).

Also provided by the present isolated is a mutant alpha 4 subunit of GABA_{A} receptor protein which has a neutral or non-basic amino acid residue substituted for the arginine residue at position 353 of the wild type mature protein and the isolated nucleic acid molecule encoding this mutated protein.

In still another aspect of the invention, there is provided a method of treating anxiety or irritability in a subject by administering to the subject an effective amount of a vector comprising an isolated nucleic acid molecule encoding a mutant alpha 4 subunit GABA_{A} receptor protein having a neutral or non-basic amino acid residue substituted for the arginine residue at position 353 of the wild type mature protein, wherein this nucleic acid molecule is operably linked to a promoter which functions in the human brain. Examples of useful promoters include but are not limited to CAM-kinase II, gamma-8 membrane-associated guanylate kinase and KCC2 (K-Cl co-transporter) promoters. This method is especially helpful in treating patients undergoing a stage such as entering or having reached puberty, suffering from pre-menstrual syndrome (PMS), entering or having reached post-partem stage, entering or having reached menopause, and suffering from chronic stress.

In still another embodiment, the present invention provides a method for identifying an antagonist of THP. The method comprises the steps of: (a) expressing α4β2δ GABA_{A} receptors in eukaryotic cells; (b) applying a drug to the eukaryotic cells of (a); (c) measuring GABA_{A} gated currents at α4 β2δ GABA_{A} receptors in the treated cells of (b); and (d) correlating a increase in outward currents recorded at α4 β2δ GABA_{A} receptors when compared to a eukaryotic cell population having THP application, with the identification of an antagonist of THP.

Also provided by the present invention are vectors comprising a subject isolated nucleic acid molecule operably liked to a promoter which functions in prokaryotic or eukaryotic cells, as well as host cells comprising such vectors.

### BRIEF DESCRIPTION OF THE DRAWINGS

FIG. 1. The neurosteroid THP decreases outward current gated by α4 β2δ GABA_{A} receptors. (a) Representative traces showing the effects of 30 nM THP (right) on current gated by 1 μM GABA (EC₇₅), under conditions of outward Cl⁻ current (inward Cl⁻ flux, upper trace) and inward current (lower trace) for two 6-containing recombinant GABA_{A} receptor subtypes. The direction of Cl⁻ current was reversed by varying internal Cl⁻ (upper trace, ECl=−70; lower trace, ECl=−30 mV), but using a constant holding potential of −50 mV. (b) Mean effects of THP on outward and inward currents in response to 1 μM GABA (upper panel) or the GABA EC₂₀ (lower panel, α4β2δ, 0.1 μM; α4β2γ2, 5 μM; α1β2γ2, 10 μM; α5β3γ2, 5 μM) from 6-7 cells for each group (*P<0.05 vs. the other receptor subtypes) (c) Current-voltage plots recorded under conditions of varying ECl (−10, 0, 20 mV) in the presence or absence of 30 nM THP. Mean±SEM for the slope conductance (gSlope) of the outward current (n=7-δ cells for each group). (d) 30 nM THP effects on current generated by a voltage ramp over 400 ms. (Leak-subtracted current is presented as the average of 3 traces). (e) Effects of the inactive 3β-OH isomer of THP on outward GABA-gated current at α4 β2δ GABA_{A} receptors (representative of 5-6 cells). (f) THP effects on desensitization of outward (upper trace) and inward (lower trace) current at α4 β2δ receptors. This effect is representative of 6 cells for each group.

FIG. 2. Arginine 353 in the α4 subunit is necessary for the direction-sensitive inhibition of α4 β2δ GABA_{A} receptors by THP. (a) Alignment of the intracellular loop of α1 and α4 (H, human; M, mouse) subunits reveals limited identity (<10%). *identical residues for all three. (The sequences for human and mouse α1 are identical.) Orange, residues to be mutated. (b) Representative traces showing the effect of 30 nM THP on GABA (10 μM)-gated current at the indicated mutated α4 β2δ GABA_{A} receptors. Basic arginine (R351 or R353) residues in the α4 subunit were mutated to a neutral glutamine (Q) and/or a basic lysine (K). (c) Effects of 30 nM THP at α4 β2δ receptors containing wild-type or mutant α4 subunits on outward GABA (1 μM)-gated current. (n=4-5 cells for each group, *P<0.05 versus wild-type α4β2δ). (d) Current-voltage plot recorded from α4-[R353Q]β2δ GABA_{A} receptors before and after THP; ECl=−4.0 mV; predicted ECl=−3.8 mV, averaged from 5 cells for each point. (e) Summary diagram. Left, amino acid sequences 316-353 within the mouse α4 loop (basic residues, blue; mutated residues, orange). The two regions of the α4 loop with consecutive basic residues (316-318 and 351-353, in blue) were mutated as a group or singly to a neutral glutamine (Q in red) or to a basic lysine (K). Right, Effects of the indicated mutation on outward and inward GABA (10 μM)-gated current are indicated, as is the GABA EC₅₀ (Mean±SEM). All mutations produced current of similar magnitude (100-200 pA; n=5-6 cells for each group).

FIG. 3. Increased expression of α4 and δ subunits on pyramidal cell dendrites of CA1 hippocampus at the onset of puberty.

(a) Immunocytochemistry of α4 (upper panel) and δ (lower panel) GABA_{A} receptor subunits in stratum radiatum of CA1 hippocampus (40× magnification). Arrows point to immunolabeling along distal portions of dendrites. Pubertal, Pub; pre-pubertal, Pre. Calibration bar applies to all four panels. The insets show background labeling taken from the ventromedial hypothalamus, a region without detectable expression of these subunits¹⁶. Representative of results from 5-6 mice for each group. (d) Electron micrograph of δ staining along the plasma membrane of the dendritic shaft (arrowhead) that is post-synaptic to an axon terminal as well as intracellularly (arrows). The long arrow points to an axon terminal that is likely to be GABAergic, based on the absence of postsynaptic density. (Representative of results from 5 pubertal mice.) (b) Western blot showing hippocampal expression of α4 and δ subunits after puberty and THP Wd compared to the pre-pubertal state. In one group, the decline in THP levels at puberty was prevented with 48 h administration of THP (10 mg kg⁻¹), Pub+THP. (c) Optical densities from Western blot results averaged from 6 hippocampi for each group normalized to the GAPDH control. *P<0.05 versus Pre-pub for all graphs. (n=3-4 animals for each group, performed in triplicate).

FIG. 4. THP inhibits tonic GABAergic current recorded from the hippocampal slice at puberty. (a) Outward current recorded from CA1 hippocampal pyramidal cells in the slice by whole-cell patch clamp (ECl=−70 mV, −50 holding potential, pipet solution, K-gluconate; bath, 200 nM gabazine to block synaptic current and 2 mM kynurenic acid to block excitatory current). Pre-pubertal, Pre-pub; pubertal, Pub; THP withdrawal, THP Wd. Inset, THP effects on the inward tonic current at puberty. (b) THP-evoked change in outward and inward tonic current, Averaged data. (mean±SEM, n=8-12 cells for each group). (c) Tight-seal cell-attached current-clamp recording³¹ of the holding potential during dendritic application of the GABA agonist gaboxadol (5 μM) to the stratum radiatum. (Representative of cells from 5 pubertal mice). (d) Perforated patch voltage-clamp recordings from the soma of a CA1 pyramidal cell of the post-synaptic response to bath applied THP. Inset, the change in access resistance determined from the current response to a 10 mV step before and after perforation. (Bath, 1 μM TTX and 1 μM GABA; also 200 nM gabazine and L-65,708, to block synaptic current and α5-GABA_{A} receptors, respectively; 10 μM CGP 55845, 5 mM TEA and 50 μM kynurenic acid to block GABA_{B} receptors, K+ channels and excitatory amino acid receptors, respectively.) (e) Averaged data, n=5 cells for each group. *P<0.01 versus pre-THP, **P<0.001 versus Pre-pub.

FIG. 5. THP increases excitability of hippocampal pyramidal cells at the onset of puberty. (a) Current-voltage plots, The difference current recorded before and after bath application of 120 μM gabazine (pipet solution, cesium-methanesulfonate; bath contains 1 μM TTX, 1 μM GABA and 50 μM L-655,708). (b) Averaged slope conductance (gSlope, assessed from −60 to −40 mV; n=5-6 cells for each group). *P<0.05 versus pre-THP, **P<0.05 versus Pre-pub. (c) Tight-seal cell-attached voltage-clamp recordings from the soma (40 mV) of CA1 hippocampal pyramidal cells³¹. (d) THP effects on spiking, averaged data. (n=5 cells for each group). *P<0.05 versus Pre-THP; **P<0.05 versus Pre-pub for all graphs.

FIG. 6. THP lowers the current threshold for spiking of pyramidal cells at the onset of puberty. (a) Whole cell current clamp recordings conducted from CA1 hippocampal pyramidal cells. Voltage responses recorded in response to increasing 0.3 nA current injection (−1 nA, initial current) for slices recorded before puberty (Pre-pub), or at puberty in wild-type (Pub) or δ^{-/-} (Pub. δ^{-/-}) mice. (The THP trace lacks the 800 pA current trace for ease of comparison.) Inset, spiking at threshold, 800 pA, pre-THP; 500 nA THP in a non-spiking pubertal cell. In some cases, Ih was blocked with 20 μM Zd 7288 (Pub+Zd 7288). Red trace, equivalent current injection, threshold for the less excitable state. Blue trace, equivalent current injection, threshold for the more excitable state.) (b) Mean±SEM averaged from 7-δ cells for each group. Current threshold to spiking, I threshold; voltage threshold to spiking, Vm threshold; spike frequency, No. of spikes; action potential amplitude, AP amplitude; action potential half-width, AP half-width. Spike frequency was assessed at the minimum current required to produce spiking in both pre- and post-THP traces. *P<0.05 versus Pre-pub.

FIG. 7. THP paradoxically increases anxiety after the onset of puberty. (a) Alterations in anxiety produced by stress or injection of THP (10 mg kg⁻¹, i.p.) are presented as a percentage change in open arm time in the elevated plus maze compared to mean values from a sham control group, identical to the experimental group (age- and genotype-matched) except for the indicated treatment (stress or THP). In order to test the role of THP release in the stress response, in some cases the inactive 3β-OH isomer of THP (stress+3β-OH-THP) or finasteride were pre-administered. Replacement THP (10 mg kg⁻¹, intraperitoneally, in oil, for three days) was also administered to prevent the decline in THP at puberty. n=6-9 mice for each group, *P<0.05 versus paired control, **P<0.05 versus Pre-pub, ***P<0.05 versus Pub restraint. (b) Open arm time (Mean±SEM) for all control groups not subjected to restraint stress.

FIG. 8 provides the nucleotide and corresponding amino acid sequence for a mouse GABA_{A} receptor Alpha-4 subunit.

FIG. 9 provides the nucleotide and corresponding amino acid sequence for human GABA_{A} receptor Alpha-4 subunit.

FIG. 10 shows in italic bold font, the change in two nucleotides of the mouse GABA_{A} receptor Alpha-4 subunit nucleotide sequence which corresponds to the mutated mouse GABA_{A} receptor Alpha-4 subunit in the examples.

FIG. 11. GABA concentration-response relationships and THP administration. GABA concentration-response curves for recombinant α4 β2δ receptors expressed in HEK-293 cells with or without application of 30 nM THP. Recordings were made under conditions of inward Cl⁻ current (a) or outward Cl⁻ current (b) produced by varying internal [Cl⁻]. Concentration-response curves were obtained using 400 ms exposure times for increasing concentrations of GABA. THP increased the efficacy of inward GABA-gated current, but reduced outward current with increasing concentrations of GABA. (n=4-5 cells for each point).

FIG. 12. THP inhibition of outward current is dependent upon GABA concentration. Current-voltage curves for a range of GABA concentrations (20 nM-100 μM, (a-e)) before and after application of 30 nM THP. THP consistently potentiated inward current at all [GABA] tested. In contrast, the magnitude of THP-induced decreases in outward current was proportional to increasing [GABA]>100 nM (b), consistent with an effect in facilitating desensitization. However, THP potentiated outward current gated by 20 nM GABA (a) at α4 β2δ GABA_{A} receptors. 1 μM GABA represents the ambient concentration at extrasynaptic α4βδ GABA_{A} receptors. (n=4-5 cells for each point)

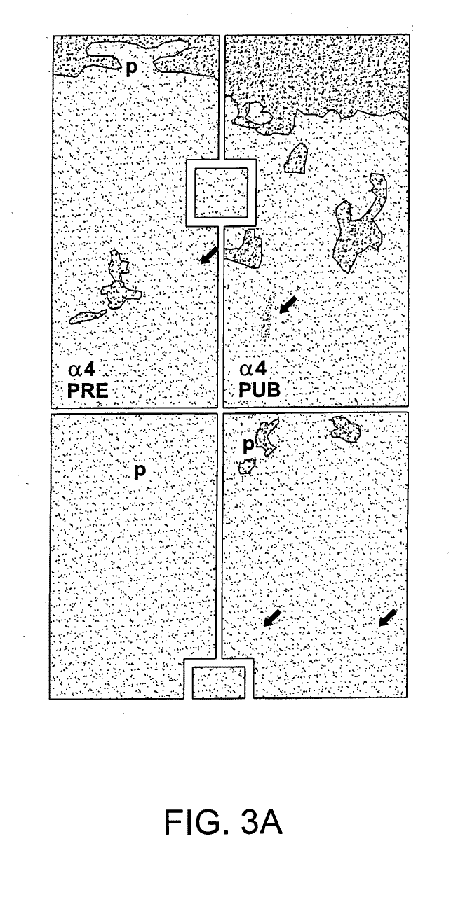

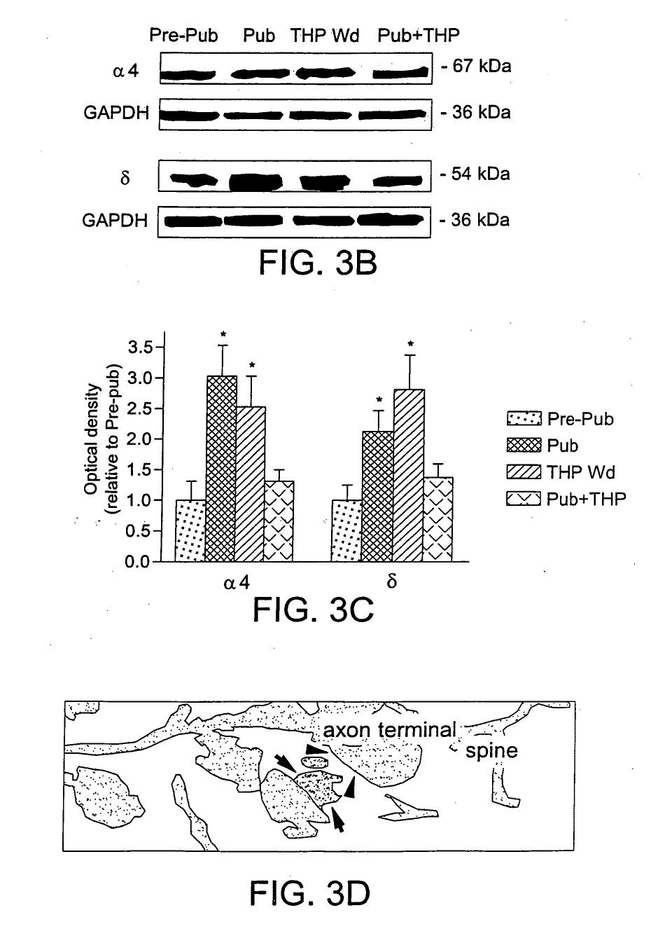

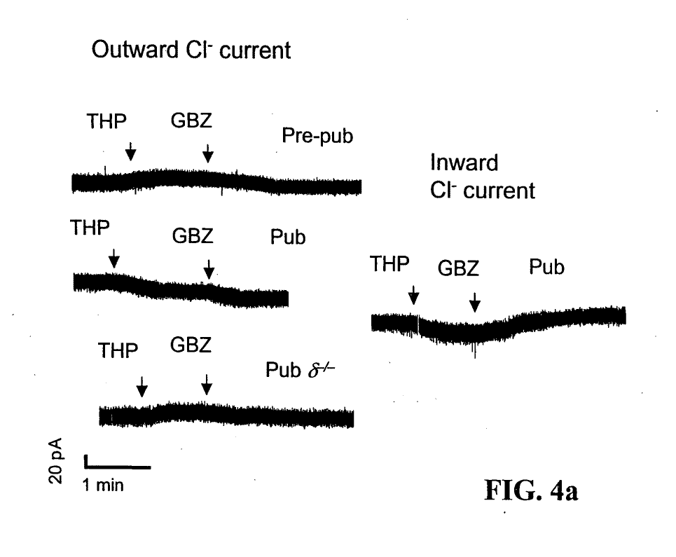

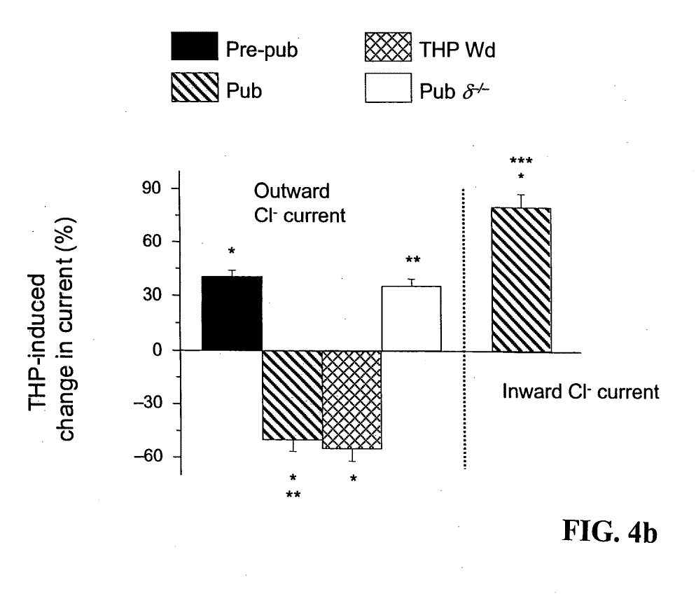

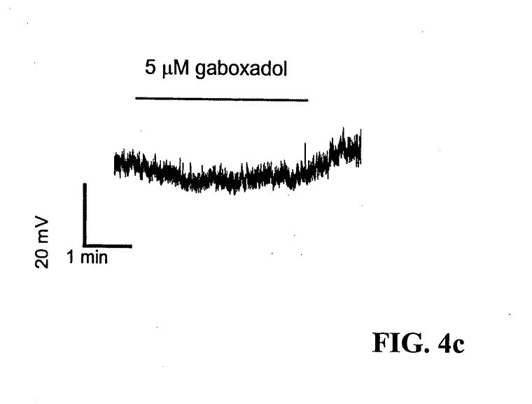

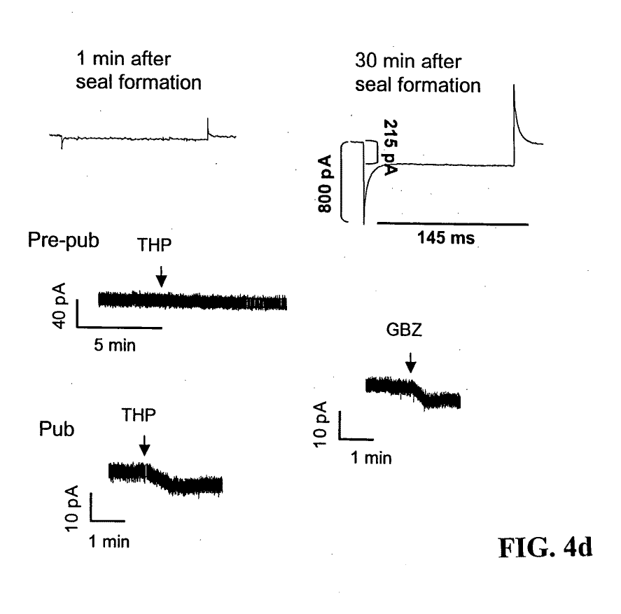

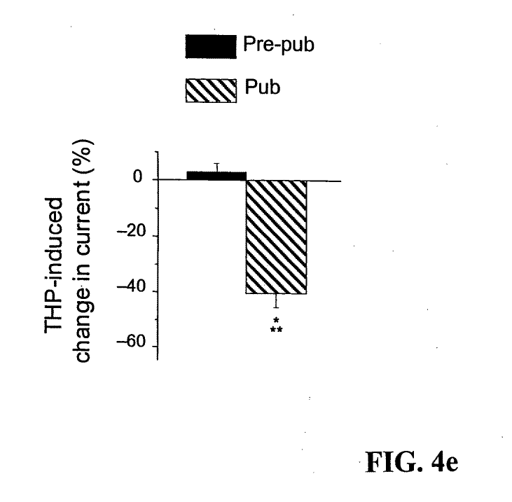

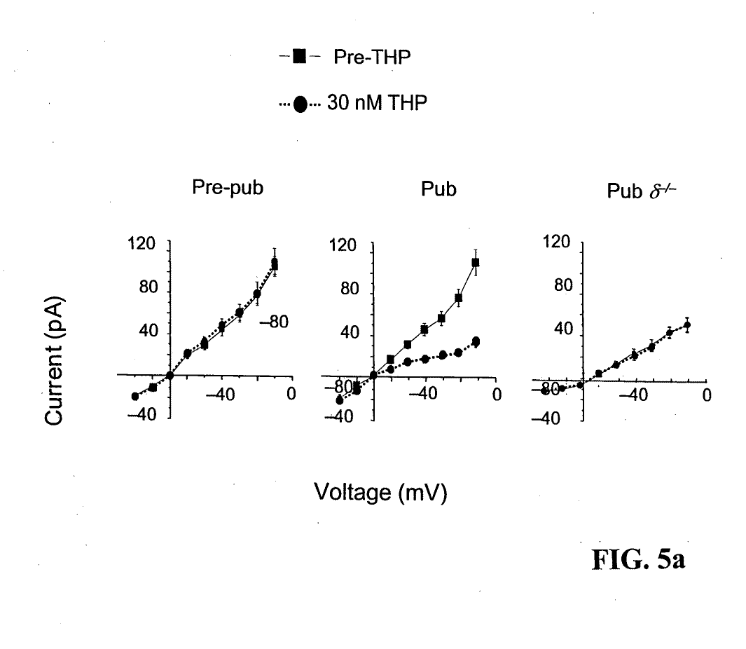

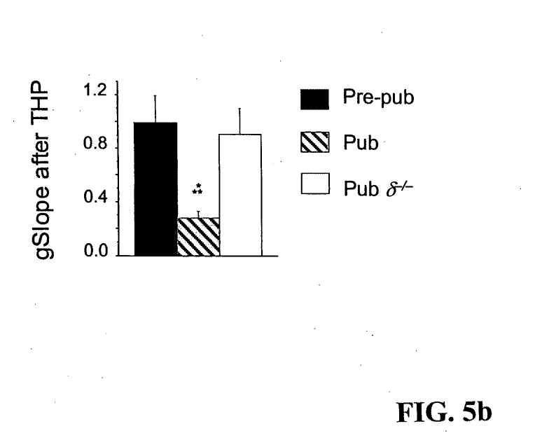

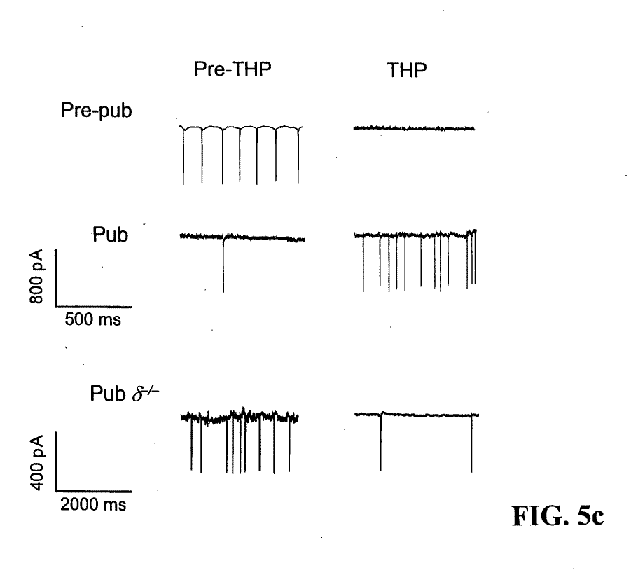

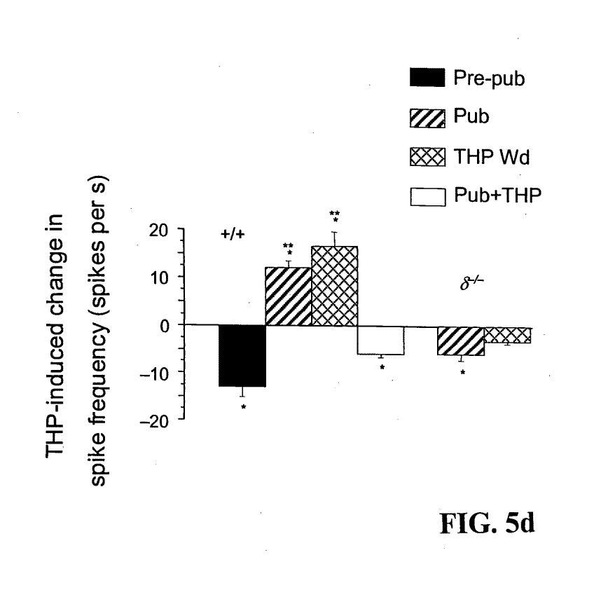

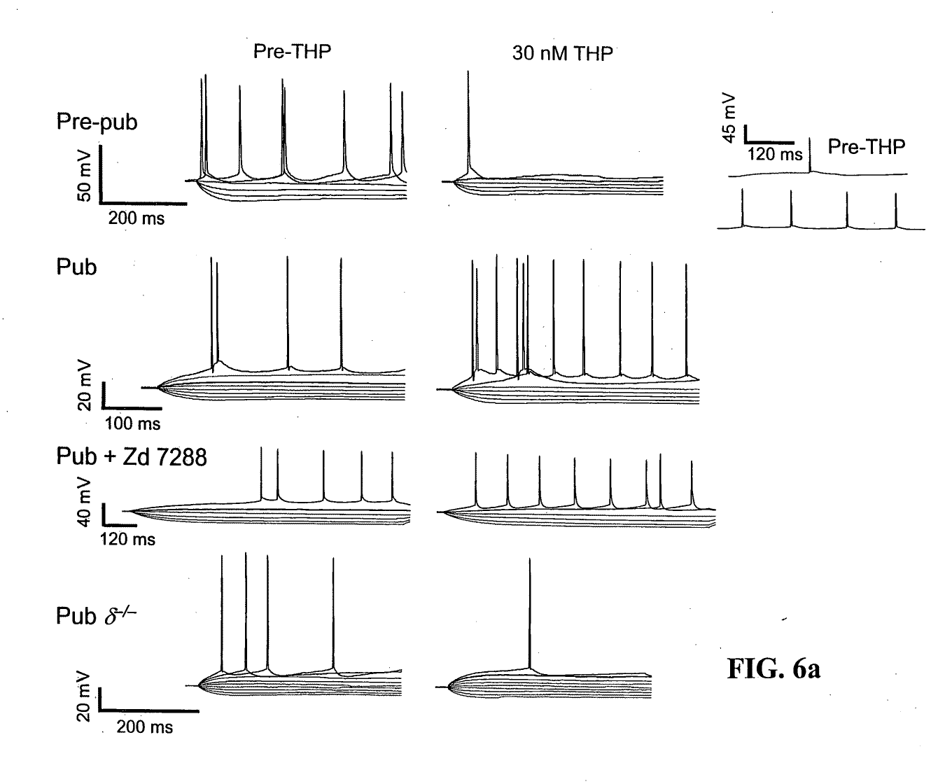

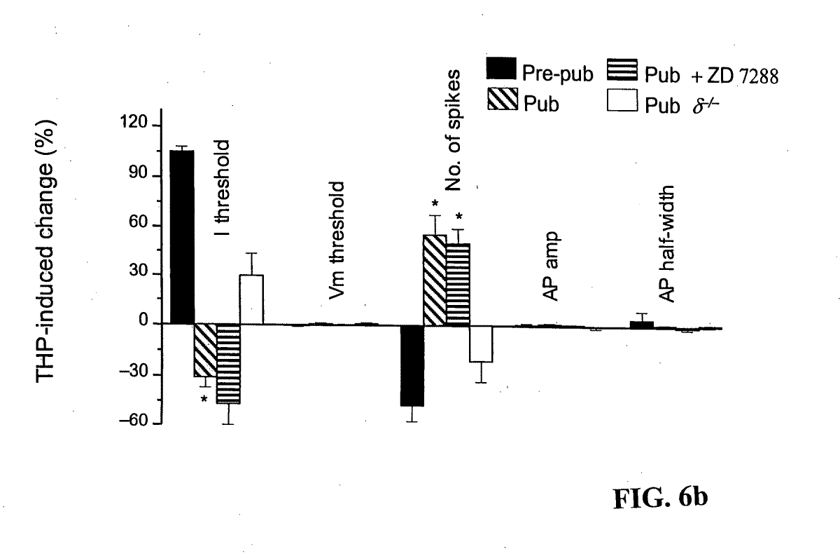

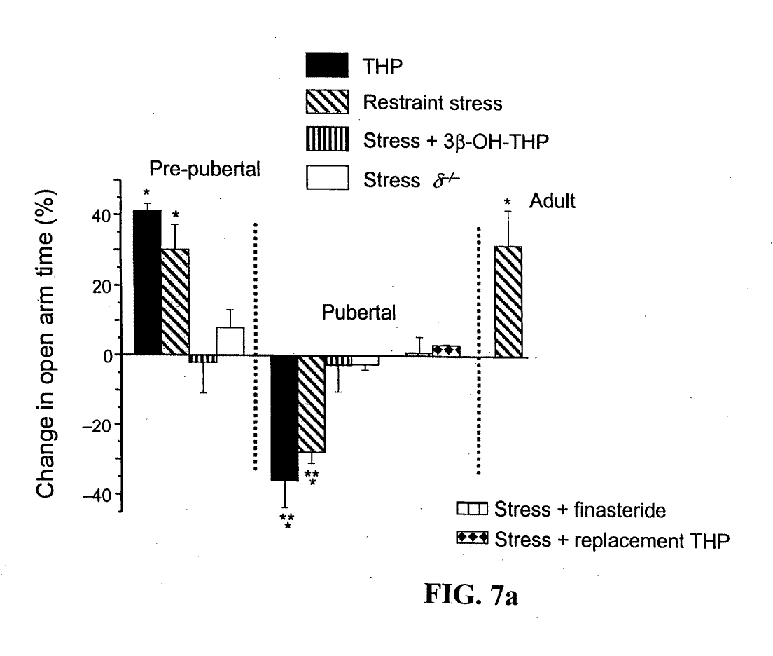

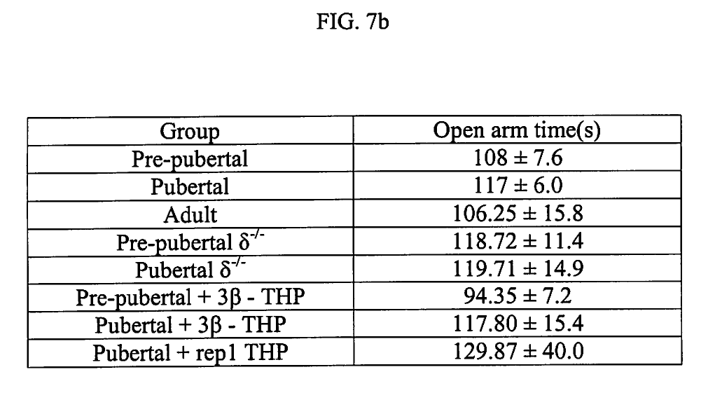

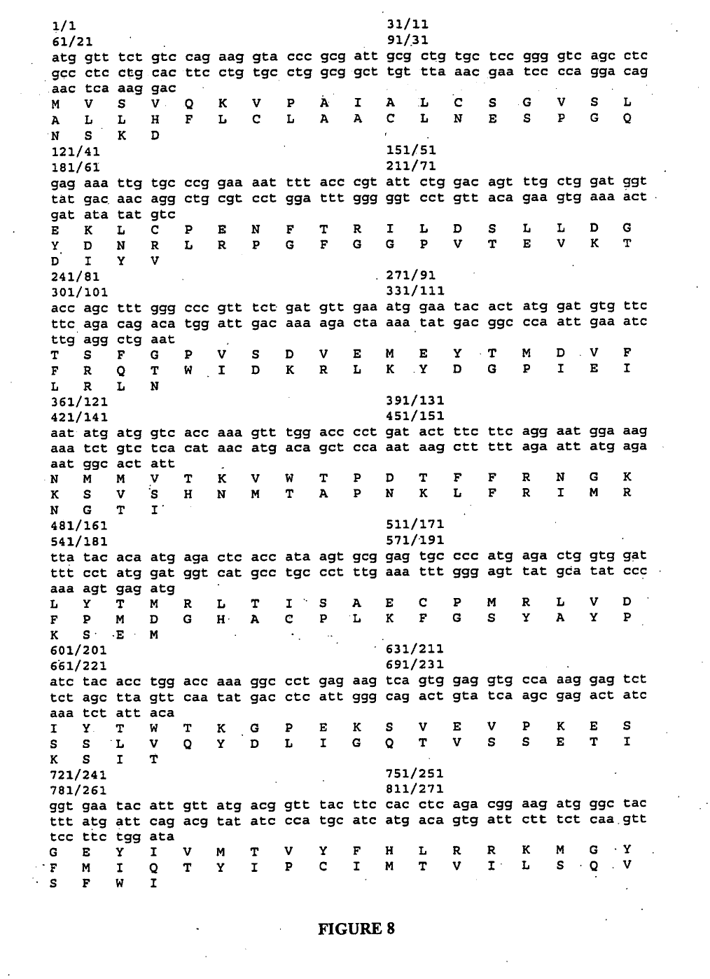

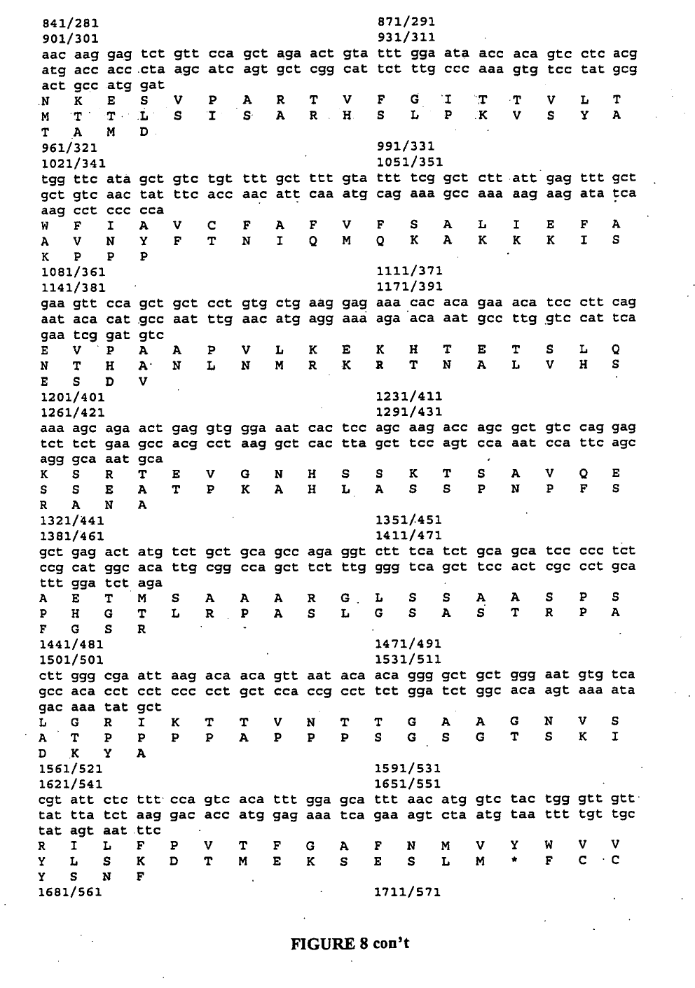

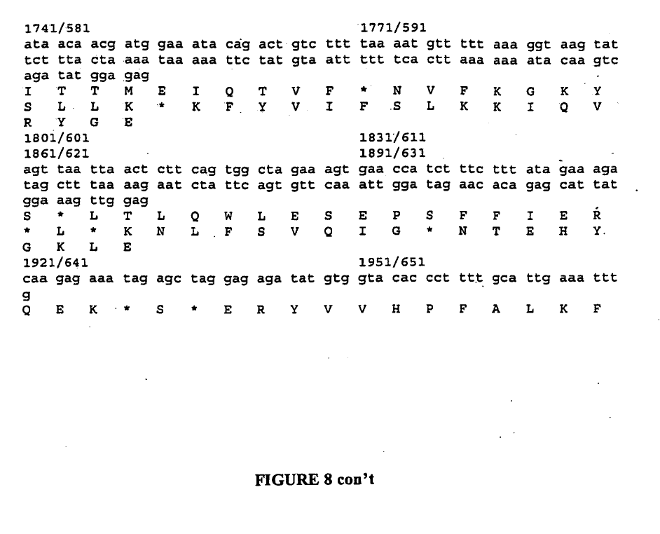

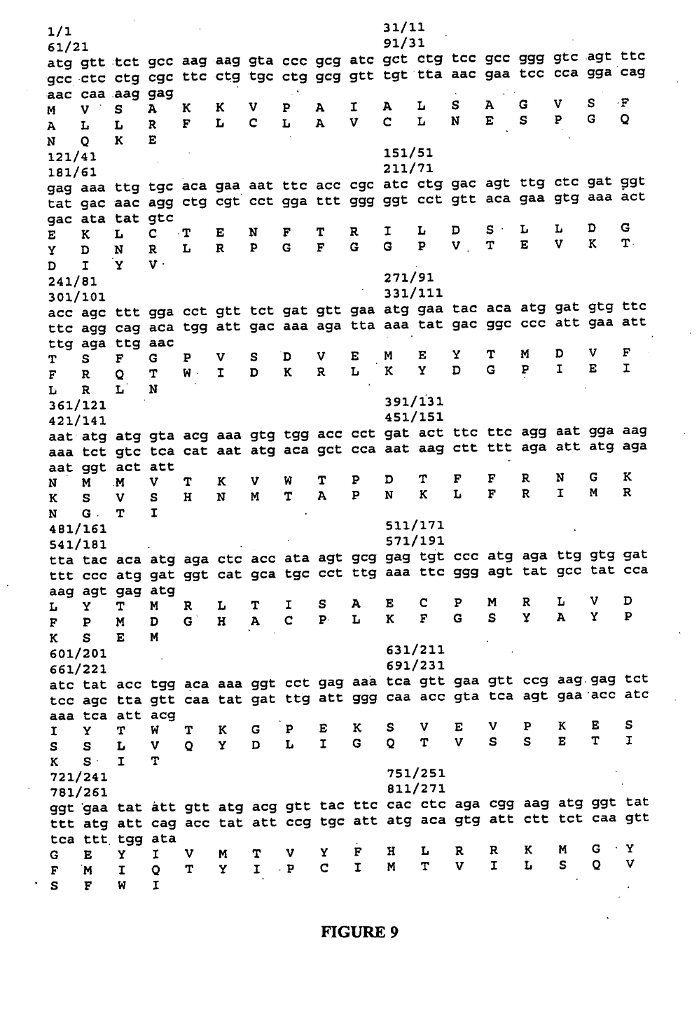

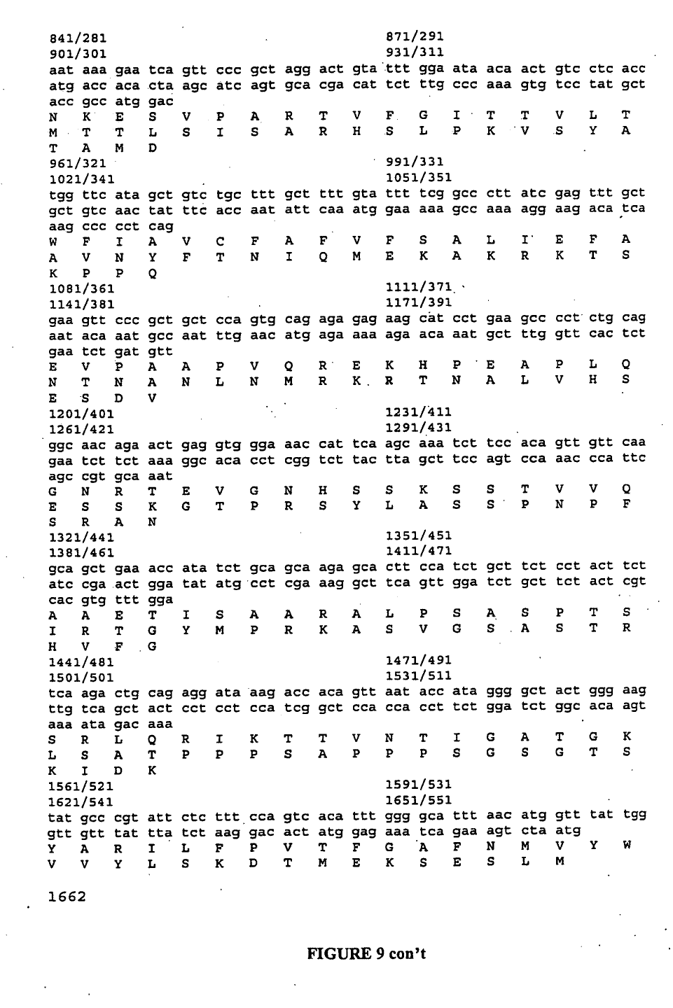

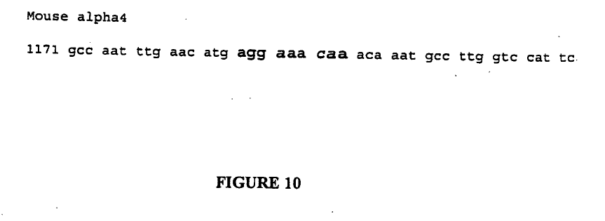

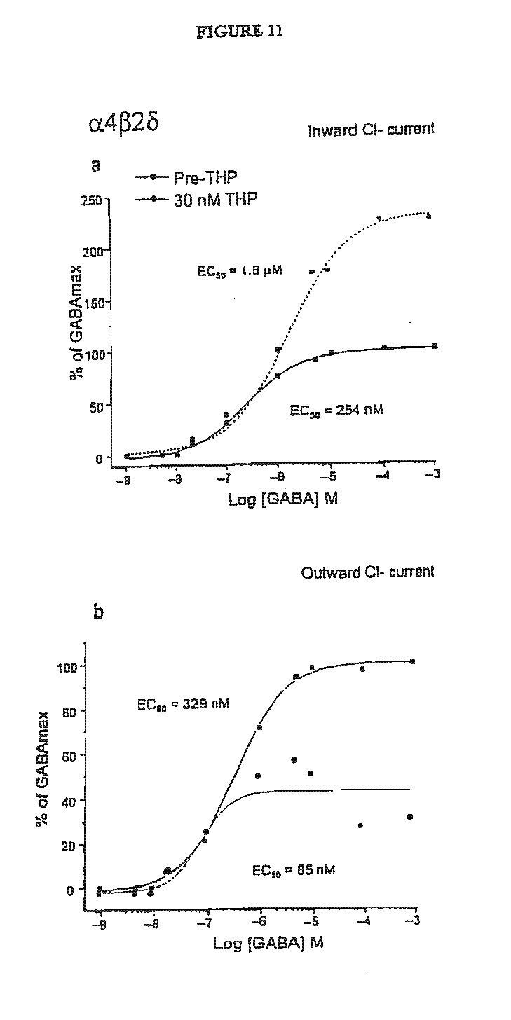

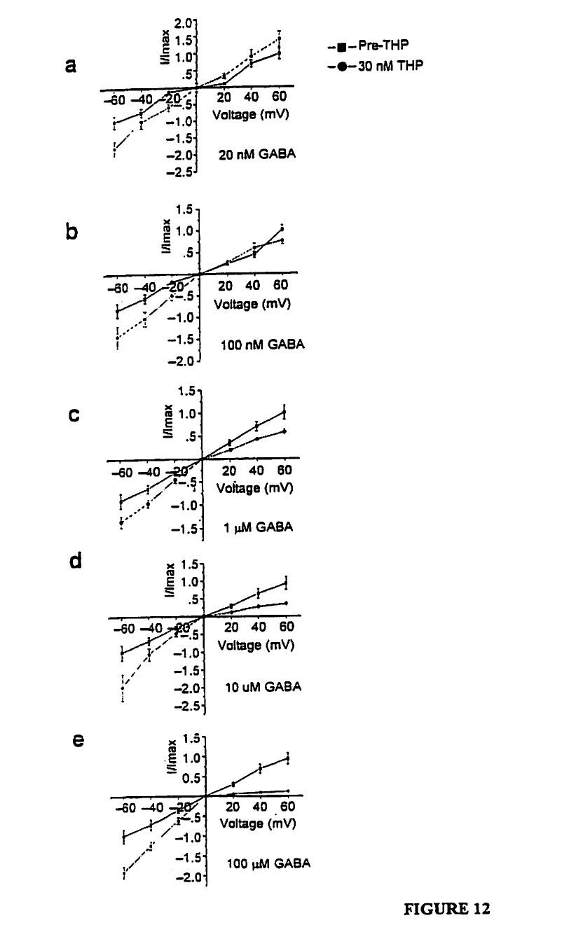

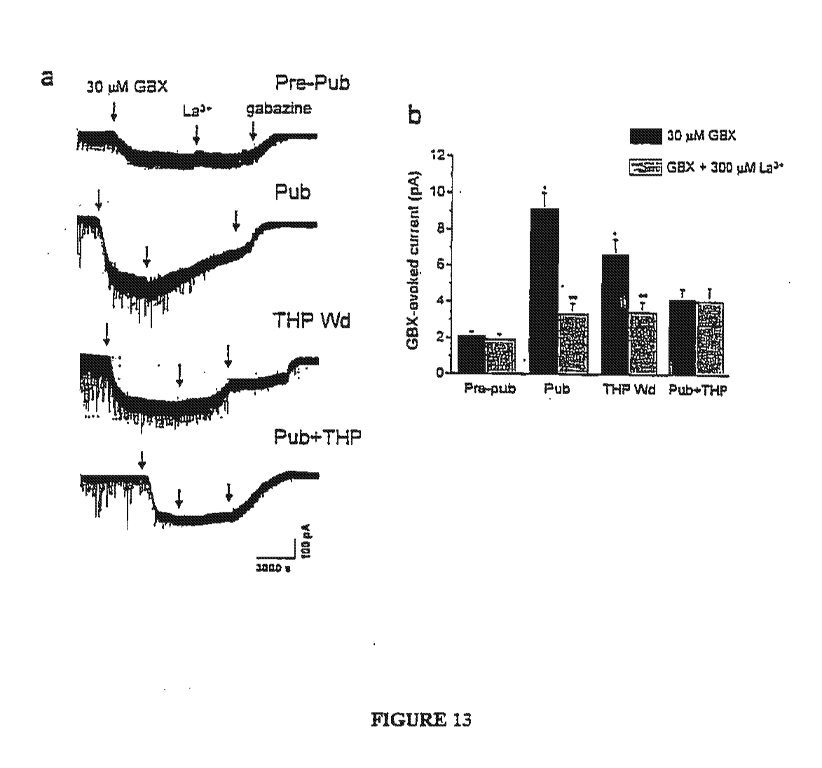

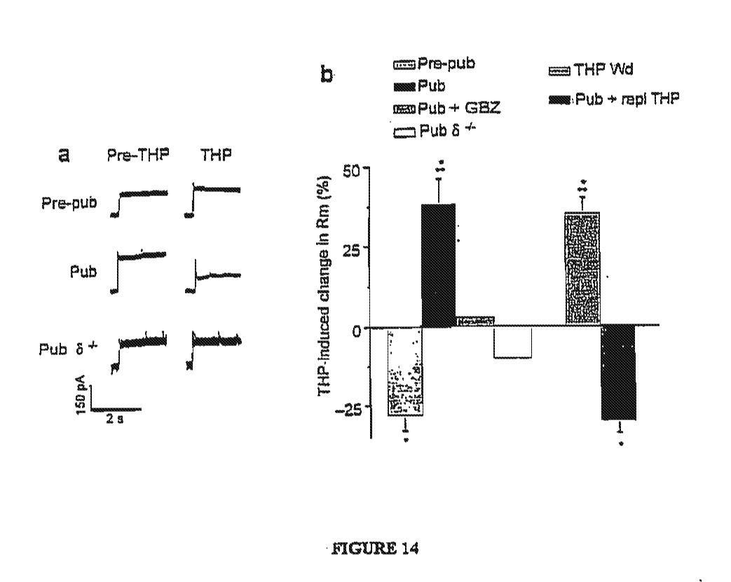

FIG. 13. Pharmacological changes in the tonic inhibitory current of CA1 hippocampus are consistent with increased expression of α4 βδ receptors after the onset of puberty. (a) The tonic GABAergic current was recorded from the soma of pyramidal cells in CA1 hippocampus with whole cell patch clamp techniques (see Methods) at a holding potential of −50 mV. Because α4β6-containing receptors have an increased sensitivity to the GABA agonist gaboxadol (GBX)¹⁶, but are uniquely inhibited by lanthanum (La³⁺)^{16,17}, the La³⁺-sensitive GBX-induced current was assessed in slices from mice after THP Wd or the onset of puberty when α4 and δ subunit expression are increased (FIG. 3). To this end, current generated by 30 μM GBX was assessed as the deflection in the holding current before and after bath application of 300 μM La³⁺ as we have described. GBX generated a significantly larger current after puberty (Pub) or after THP Wd than in slices from pre-pubertal (Pre-pub) mice, and this current was reduced 50-70% by La³⁺. The profile in pubertal mice treated with replacement THP was similar to that in pre-pubertal mice, suggesting that changes occurring after the onset of puberty are due to a withdrawal from THP. (a) Representative traces; (b) Mean±SEM. n=5-6 cells for each group. *P<0.05 vs. Pre-pubertal values, **P<0.05 vs. GBX alone. GBX, gaboxadol.

FIG. 14. THP increases the input resistance of hippocampal pyramidal cells at the onset of puberty. (a) Current response to a 10 mV step (−50 to −40 mV), recorded from the soma with whole cell voltage clamp techniques before and after 30 nM THP in slices from pre-pubertal (Pre-pub) or pubertal (Pub) wild-type or δ^{-/-1} mice. (b) Mean±SEM. THP increased the input resistance (Rm) at puberty, assessed from the current response to two 10 mV steps (60 to −40 mV). This effect was prevented with pre-application of the GABA_{A} blocker 120 μM gabazine (Pub+GBZ) and was not seen in slices from δ^{-/-}mice, suggesting that the change in input resistance was mediated by 6-containing GABA_{A} receptors. In contrast, THP decreased input resistance, when assessed before puberty (Pre-pub). Similar steroid-induced increases in input resistance were produced after THP withdrawal (THP Wd). In addition, administration of replacement THP to prevent the decline in THP occurring at puberty (Pub+repl THP) prevented the steroid-induced change in input resistance. (n=8-10 cells for each group, *P<0.05 vs. pre-THP values; **P<0.05 vs. Pre-pubertal values.).

### DETAILED DESCRIPTION OF THE INVENTION

In accordance with the present invention, it has been surprisingly found that THP (3α-OH-5α[β]-pregnan-20-one or allopregnanolone), a steroid released by stress, increases anxiety in pubertal female mice, a reversal of its well-known anxiety-reducing effects in adults. This surprising effect of THP is due to inhibition of α4 βδ GABA_{A} receptors. Anxiety is regulated by GABAergic inhibition in limbic circuits. Although this inhibition is increased by THP before puberty and in adults, it has now been found that THP reduces tonic inhibition of CA1 hippocampal pyramidal cells at puberty, leading to increased excitability. Normally, α4 βδ GABA_{A} receptors are expressed at very low levels, but at puberty, their expression is increased in CA1 hippocampus where they generate outward currents.

Also in accordance with the present invention, it has been found that THP also decreases outward current at recombinant α4 β2δ receptors, an effect dependent on arginine 353 in the α4 subunit, a putative Cl⁻ modulatory site. Thus, inhibition of α4β2δ GABA_{A} receptors by THP provides a mechanism for anxiety at puberty and the present invention provides methods of reversing the stress and anxiety in female subjects entering or undergoing puberty as well as a number of other states such as pre-menstrual syndrome (PMS), post partem stage, or menopause.

In accordance with the present invention, a specific site on the brain molecule GABA-A receptor, has been identified which site leads to excitability in the central nervous system. This discovery has special relevance for fluctuations in naturally occurring steroids, including the periods of puberty, premenstrual syndrome, menopause and post-partum, as well as chronic stress.

In a first embodiment of the invention, there is provided a method for inhibiting or treating anxiety or irritability in a subject, said method comprising: administering to the subject an effective amount of an antagonist of allopregnanolone (THP). An example of an antagonist of THP is 3βOH-5α[β]-pregnan-20-one. In accordance with the present invention, the subject may be entering or may be in a period or stage such as puberty, pre-menstrual syndrome (PMS), post-partum, menopause, or chronic stress.

Methods for identifying additional antagonists of THP are also provided. The method comprises the steps of: (a) expressing α4 β2δ GABA_{A} receptors in eukaryotic cells; (b) applying a drug to the eukaryotic cells of (a); (c) measuring GABA_{A} gated currents at α4 β2δ GABA_{A} receptors of (b); and (d) correlating a increase in outward currents recorded at α4 β2δ GABA_{A} receptors when compared to a eukaryotic cell population having THP application, with the identification of an antagonist of THP.

In another embodiment of the invention, there is provided a method for treating anxiety or irritability in a subject entering or in a period or stage such as puberty, pre-menstrual syndrome (PMS), post-partem, menopause or chronic stress. The method comprises administering to the subject an effective amount of a regulator which decreases expression of the α₄β_{2δ} subunit of GABA_{A .}

An example of a regulator which decreases expression of the α₄β_{2δ} subunit of GABA_{A} is gabadoxbol (THIP). Methods for identifying other drugs which decrease expression of the α₄β₂δ subunit of GABA_{A} are described in copending U.S. patent application Ser. No. 10/566,559, the entirety of which is incorporated by reference herein as if fully set forth.

In still another aspect of the present invention, it has been found that mutation of a positively charged arginine (R) at position 353 to a neutral or non-basic residue in the α4 subunit of GABA_{A} receptors prevents the steroid-induced reduction in outward current of α4 βδ GABA_{A} receptors. Mutation of R353 to another basic residue, e.g., lysine (K) does not prevent THP inhibition of outward current. Thus, the present invention provides an isolated mutated protein comprising the amino acid sequence of GABA-_{A} receptor Alpha-4 subunit receptor having a neutral or non-basic residue at position 363. Examples of neutral amino acids which may reside at position 353 include e.g., alanine (neutral), asparagine (neutral), aspartic acid (acidic), cysteine (neutral), glutamic acid (acidic), glutamine (neutral), glycine (neutral), isoleucine (neutral), leucine (neutral), methionine (neutral), phenylalanine (neutral), proline (neutral), serine (neutral), threonine (neutral), tryptophan (neutral), tyrosine (neutral), or valine (neutral).

Nucleotide sequences for the various GABA_{A} receptor subunits, are known and widely available. For example, nucleotide sequences for mouse (51) or human α₄ (52), may be used to generate the mutant GABA-_{A} receptor Alpha-4 subunit receptor having a neutral or non-basic residue at position 353 of the mature protein. Methods for generating mutations at a specific amino acid site, e.g., 353, are well known and preferably involve use of a commercially available kit for site-directed mutagenesis such as the Quick-Change Mutagenesis Kit I or II (Stratagene Inc., La Jolla, Calif.). Sequencing of end-products is preferably performed to verify the site-directed mutation.

The present invention further provides an isolated amino acid sequence for a mutated GABA-_{A} receptor Alpha-4 subunit receptor having a neutral or non-basic residue at position 353. Thus for example, in FIG. 9, the nucleotides at positions corresponding to amino acid 353 of the GABA-_{A} receptor Alpha-4 subunit may be altered by site directed mutagenesis to encode a neutral or non-basic residue. In FIG. 9, the nucleotides encoding the 353 residue of the mature protein reside at positions 1171-1173 due to FIG. 9 showing the signal sequence at amino acids 1 through 37. In FIG. 9, nucleotides 1171-1173 encode an arginine (R) shown in bold. By changing the ag of the aga codon to an ca, thereby making an caa codon, the corresponding amino acid residue is changed from an arginine (wild type) to a glutamine (Q) residue. Using the genetic code, the skilled artisan is aware of the many different codons which may be used to substitute a neutral or non-basic residue at position 353 of the mature protein. Fragments of the mutant GABA-_{A} receptor Alpha-4 subunit having a neutral or non-basic reside at position 353 are also provided by the present invention.

Also provided by the present invention is a nucleotide sequence encoding a mutant GABA-_{A} receptor Alpha-4 subunit having a neutral or non-basic reside at position 353. Fragments of the nucleotide sequence encoding a mutant GABA-_{A} receptor Alpha-4 subunit having a neutral or non-basic reside at position 353 are also provided.

Site-specific or site-directed mutagenesis allows the production of peptide variants through the use of specific oligonucleotide sequences that encode the DNA sequence of the desired mutation plus a sufficient number of adjacent nucleotides, to provide a primer sequence of sufficient size and sequence complexity to form a stable duplex on both sides of the deletion junction being traversed. Typically, a primer of about 20 to 30 nucleotides in length is preferred, with about 5 to 10 residues on both sides of the junction of the sequence being altered. The technique of site-directed mutagenesis is well known in the art, as exemplified by publications such as Adelman et al., DNA 2:183 (1983), which is incorporated herein by reference as if fully set forth. As will be appreciated, the mutagenesis technique typically employs a phage vector that exists in both a single-stranded and double-stranded form. Typical vectors useful in site-directed mutagenesis the M13 phage (Messing et al., Third Cleveland Symposium on Macromolecules and Recombinant DNA, Editor A. Walton, Elsevier, Amsterdam (1981)). These phage are commercially available and their use is well known to those skilled in the art. Alternatively, plasmid vectors that contain a single-stranded phage origin of replication (e.g., Veira et al., Meth. Enzymol. 153:3 (1987)) may be employed to obtain single-stranded DNA.

In general, site-directed mutagenesis in accordance herewith is performed by first obtaining a single-stranded vector that includes within its sequence a DNA sequence that encodes the GABA-_{A} Alpha-4 receptor subunit (or peptide). An oligonucleotide primer bearing the desired mutated sequence is prepared, generally synthetically (e.g., Crea et al., Proc. Natl. Acad. Sci. (USA) 75:5765 (1978). This primer is annealed with the vector comprising the single-stranded protein-coding sequence and is subjected to DNA-polymerizing enzymes such as E. coli polymerase I Klenow fragment to complete the synthesis of the mutation-bearing strand. Thus, a mutated sequence and the second strand bears the desired mutation. This heteroduplex vector is then used to transform appropriate cells (such as JM101 cells) and clones are selected that include recombinant vectors bearing the mutated sequence arrangement.

After such a clone is selected, the mutated protein region may be removed and placed in an appropriate vector for protein production, generally an expression vector of the type that may be employed for transformation of an appropriate host.

While the present invention is directed primarily to human GABA-_{A} receptor Alpha-4 subunit, it is to be understood that homologues of GABA-_{A} receptor Alpha-4 subunit from other species are intended within scope of the present invention. In particular, the GABA-_{A} receptor Alpha-4 subunit from other species (DNA or protein), may be used for the same purposes as human GABA-_{A} receptor Alpha-4 subunit. See e.g., FIG. 8 which provides the nucleotide and corresponding sequence for GABA-_{A} receptor Alpha-4 subunit from mouse.

The present invention also provides fragments or peptides of GABA-_{A} receptor Alpha-4 subunit which include the mutation of residue 353 to a neutral or non-basic amino acid. Such fragments may be produced using enzyme digestion. Peptides may be produced using well-known synthetic methods for the synthesis of polypeptides of desired sequence on solid phase supports and their subsequent separation from the support. Methods for solid phase peptide synthesis are well-described in the following references, incorporated by reference herein as if fully set forth: Merrifield, B., J. Amer. Chem. Soc. 85:2149-2154 (1963); Merrifield, B., Science 232:341-347 (1986); Wade, J. D. et al., Biopolymers 25:S21-S37 (1986); Fields, G. B., Int. J. Peptide Prot. Res. 35:161 (1990); MilliGen Report Nos. 2 and 2a, Millipore Corporation, Bedford, Mass., 1987). For example, the more classical method, “tBoc method,” or the more recent improved “F-moc” technique may be used (Atherton, E. et al., J. Chem. Soc. Perkin Trans. 1:538-546 (1981)).

In still another aspect of the invention, there is provided a method of treating anxiety or irritability in a subject, said method comprising administering to the subject an effective amount of a vector comprising a subject isolated nucleic acid molecule operably linked to a promoter which functions in the human brain. In accordance with the present invention, a subject is undergoing a stage selected from the group consisting of: entering or having reached puberty, suffering from pre-menstrual syndrome (PMS), entering or having reached post-partem stage, and entering or having reached menopause.

Preferably, the promoter which functions in the brain is specific for a protein localized on principal cells of the limbic system. Examples of such promoters include but are not limited to: CAM-kinase II, gamma-8 membrane-associated guanylate kinase, and KCC2 (K-Cl co-transporter). Such promoters are known and publicly available.

Also provided by the present invention are vectors comprising a subject isolated nucleic acid molecule operably linked to a promoter which functions in prokaryotic or eukaryotic cells, as well as a prokaryotic and/or eukaryotic host cells harboring such vector. Any of the well known and publicly available vectors which replicate in prokaryotic or eukaryotic cells may be employed in practicing the present invention. Prokaryotic host cells e.g., bacterial cells as well as eukaryotic host cells, e.g., yeast, insect, and mammalian cells such as CHO cells and green monkey kidney cells, are widely known and available for practicing the present invention.

The following examples further illustrate the invention and are not meant to limit the scope thereof.

### Example 1

### Materials and Methods

Animal subjects. Prepubertal and pubertal female C57BL6 mice (3½-6 weeks old, +/+ and δ^{-/-1}) were housed in a reverse light:dark cycle (12:12). Both sets of mice were originally supplied by G. Homanics (Univ. of Pittsburgh), and were bred on site, with +/+ mice supplemented from Jackson Laboratories (Bar Harbor, Me.). Initially, age-matched+/+littermates of the δ^{-/-}were used as controls, but because they were indistinguishable from C57BL6, data from both groups were pooled. Genotyping of the tails verified that mice were homozygous δ^{-/-}. Some animals (+/+ or δ^{-/-}) were injected with finasteride (1,(5α)-androstene-4-aza-3-one-N-tert-butyl-17β-carboxaminde, Steraloids, 50 mg kg^{, -1} intraperitoneally, 3 days) to block THP synthesis² or oil vehicle on a daily basis 1-1.5 h before dark onset, when THP levels increase, and tested 30 min. later. The onset of puberty was determined by vaginal opening, and pubertal mice tested on the day of first metestrus, identified by vaginal morphology and verification of first estrus the previous day. Mice with vaginal smears representative of diestrus were excluded from the study. Individual stages of puberty were not distinguished, and pre-pubertal mice were used before any visible signs of pubertal development, assessed by development of the peri-vaginal area. In some cases, pubertal mice were injected with replacement THP (3α-OH-5β-pregnan-20-one, 10 mg kg⁻¹, intraperitoneally in oil) for three days before testing. Procedures were in accordance with the SUNY Downstate Institutional Animal Care and Use Committee.

Radioimmunoassay for 3α,5α-THP. Hippocampal levels of 3α,5α-THP were assessed by radioimmunoassay (RIA) by C. A. Frye (SUNY Albany) during the nocturnal surge³, 1 h after dark onset, according to previously published methods⁴. Following a methanol extraction, the lipophilic fraction was chromatographed on Sepak columns. The RIA was accomplished with a specific antibody to 3α,5α-THP (921412-5)₅ (purchased from R. Purdy, Veteran's Medical Center, La Jolla, Calif.) at a concentration of 1:5000 and incubated overnight with [³H] steroid at 4° C. Separation of bound and free 3α,5α-THP was accomplished by the rapid addition of dextran-coated charcoal, followed by centrifugation. Sample tube concentrations were calculated using the logit-log method⁶, interpolation of the standards, and correction for recovery. The minimum detectable limit of the assay was 50 pg. The intra-assay and inter-assay coefficients of variance were 0.12 and 0.15.

Western blot: Procedures were performed on hippocampal membranes at protein concentrations in the linear range (5-10 μg), as we have described⁷. Following preparation of crude hippocampal membranes, individual protein concentrations were assessed using the Bradford assay. Equal amounts of protein were loaded onto a 10% NuPage Bis-Tris gel, and electrophoresed, followed by transfer of proteins to nitrocellulose membranes (Invitrogen). Following a 1 h block with 5% non-fat dry milk, membranes were incubated overnight at 4° C. with a 1:15,000 (a4⁷), 1:5000 (6) and 1:100,000 (glyceraldehyde-3-phosphate dehydrogenase, GAPDH) dilution of the antibody, followed by a 1:5,000 dilution of horseradish peroxidase-conjugated donkey anti-rabbit IgG (a4, δ) or goat anti-mouse IgG (GAPDH) (Sigma). α4 (67 kDa) and δ (54 kDa) bands were detected with enhanced chemiluminescence (Pierce Supersignal West Femto substrate). (The δ antibody was a generous gift from W. Sieghart.) Optical densities were quantified with One-Dscan software from a scanned image. Results were standardized to a GAPDH (36 kDa) control and are presented as a ratio relative to the pre-pubertal values.

Preparation of brains for light and electron microscopy. Female mice were transcardially perfused under deep anesthesia with the following solutions: (1) 100-300 ml of heparinized saline over a 1 min period; and (2) 4% paraformaldehyde in 0.1 M phosphate buffer (PB), set at pH 7.4, over a 15 min period^{8,9}. Brains were post-fixed for 24 hours before sectioning. Sections containing the hippocampus, and in some cases, the ventromedial hypothalamus (VMH, negative control), were prepared using a Vibratome, set at a thickness of 40 μm in the coronal plane. Sections were stored at 4° C. in saline (0.9% NaCl), buffered by 0.01M phosphate salts (pH 7.4, PBS) and with 0.05% sodium azide to retard bacterial growth.

Immunocytochemistry. Immunocytochemical labeling of receptor subunits was achieved by the pre-embed DAB (3,3′-diaminobenzidine HCl) procedure, as described^{8,9}. Sections representing each animal of the groups, pre-pubertal and pubertal, were processed strictly in parallel, so as to minimize variability arising from differences in the concentration or quality of immunoreagents or in the incubation period of the immunoreagents. Sections containing the hippocampus and ventromedial hypothalamus were incubated in PBS containing 1% H₂O₂ to reduce background staining. These were incubated overnight at room temperature in PBS containing the antibody directed against the α4 subunit (SC-7355¹⁰, Santa Cruz Biotechnology) at a dilution of 1:20 or 1:100. Another set of semi-adjacent sections were incubated in PBS containing δ antibodies (a generous gift from W. Sieghert¹¹) at a concentration of 1:1000 (0.2 μg ml⁻¹) or 1:800. One-percent bovine serum albumin (BSA) and 0.05% sodium azide were added to these antibody dilutions to diminish nonspecific labeling. Sections were incubated in biotinylated secondary antibodies (anti-goat IgG, 1:1000 for the α4-subunit antibody and anti-rabbit IgG for the sections incubated in the anti-□subunit antibody, both from Vector), then in the ABC Elite kit mixture, followed by a reaction of horseradish peroxidase (HRP) using DAB plus H₂O₂ as substrate. The sections immunolabeled for the a subunit were incubated briefly in 0.1% osmium tetroxide to intensify the HRP-DAB immunolabeling. For light microscopy, staining in the CA1 field and elsewhere were visualized using a 20× or 40× objective. For electron microscopy, free-floating sections were post-fixed using 1% glutaraldehyde in PBS, then with 1% osmium tetroxide in 0.1M PB, then infiltrated using Embed812. Vibratome sections were ultrathin-sectioned at a setting of 70-90 nm. The surface-most regions of the vibratome sections were probed for the presence of HRP-DAB reaction product using the JEOL transmission electron microscope at magnifications ranging from 750× to 40,000×. Further details of the electron microscopic procedure appear elsewhere⁸.

Recombinant receptors: Transfection—Plasmids obtained from S. Vicini (rat α1, α5, β2, β3 and γ2), P. Whiting (human α4 and δ) and N. L. Harrison (mouse α4) were propagated in E coli DH5α and prepared using Qiagen Maxi- or Midi-prep kits. (Rat, mouse and human α1, β2, β3 and γ2 subunits are identical or nearly identical.) All receptors were transfected at a 1:1:1 ratio, except α1β2γ2 (1:1:5)¹², α4βδ (5:1:5)¹³ and α4β2γ2 (5:1:1). HEK-293 cells were maintained in medium (DMEM:Ham's F-12 1:1) supplemented with 10% fetal calf serum at 37° C. in a humid 5% CO₂ atmosphere. Cells were transfected using calcium phosphate or Polyfect (Qiagen) and co-transfected with enhanced green fluorescent protein (ratio of DNA to eGFP was 6:1 or 10:1 depending on the DNA concentration) for visualization. α4-containing GABA_{A} receptors required 2-3 d for maximal levels of expression; all other receptors were recorded 1-2 d after transfection. Because mouse α4 and human α4-containing receptors yielded the same results, the currents were averaged together.

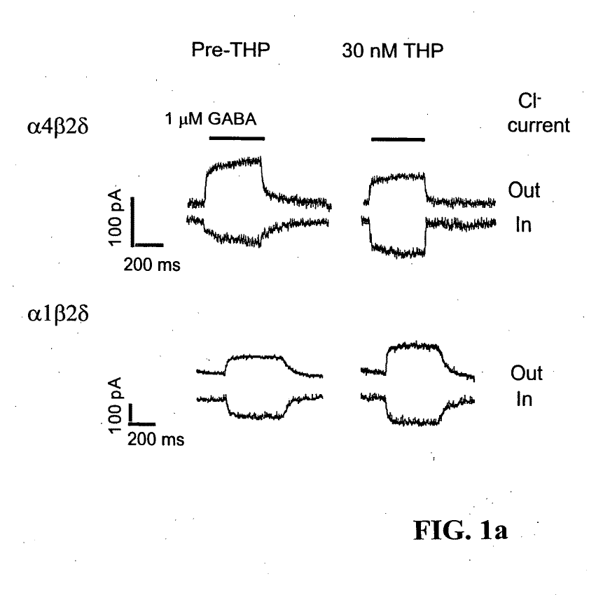

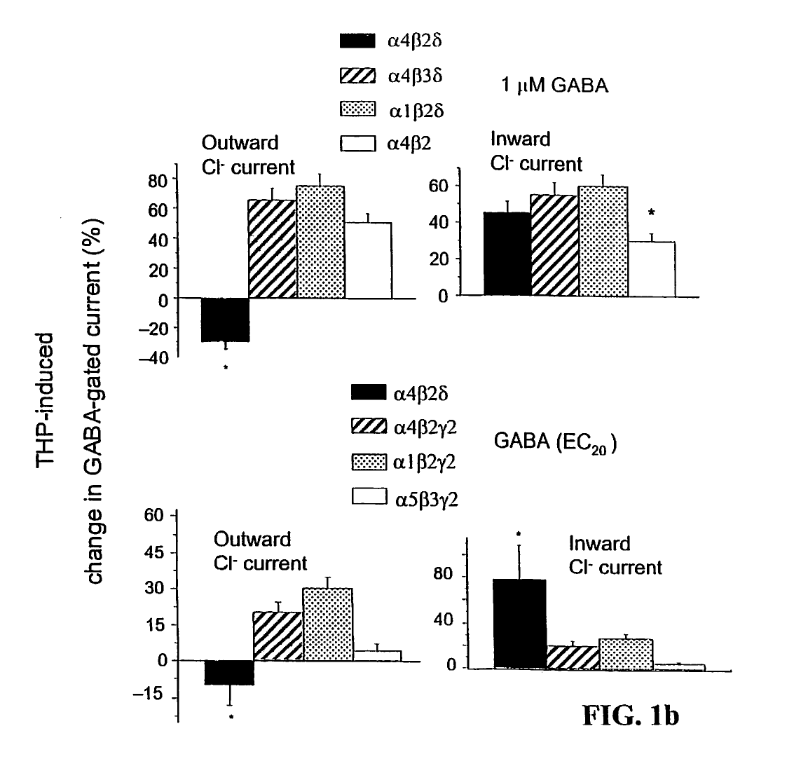

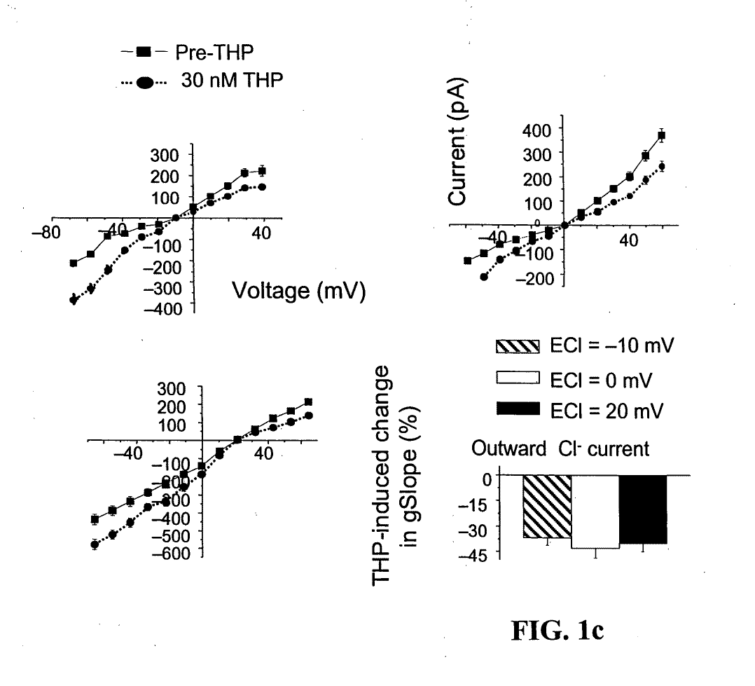

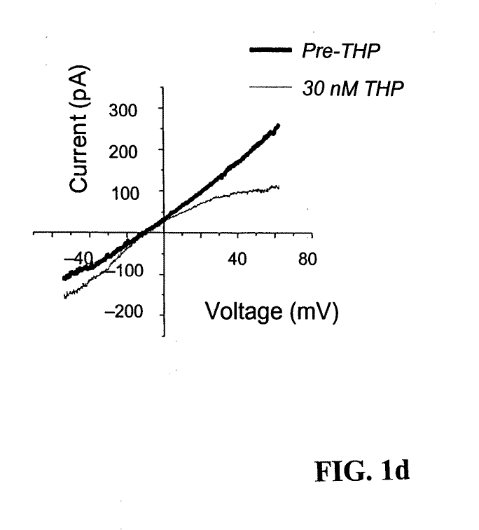

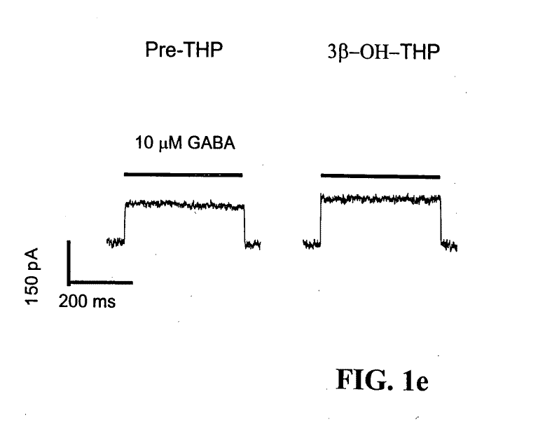

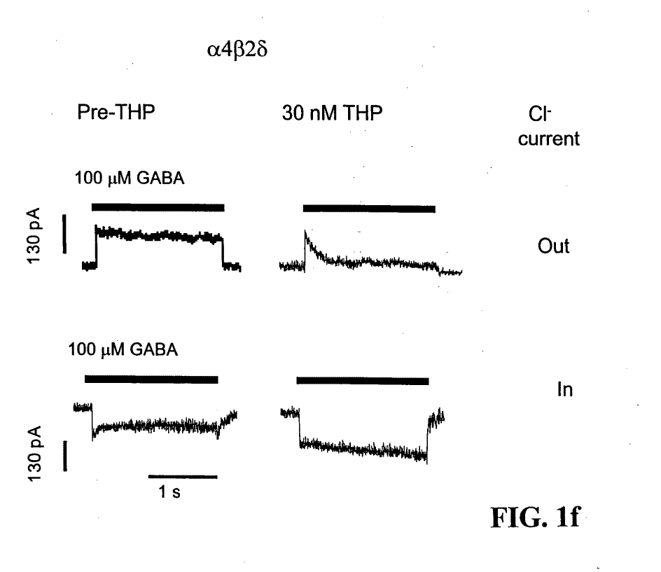

Whole cell patch clamp recording—GABA-gated currents were recorded at room temperature (20-22° C.) at a holding potential of −50 mV¹⁴. The pipet solution contained (in mM): N-methyl-D-glucamine chloride 120, Cs4BAPTA 5 (Calbiochem), Mg-ATP 5, and an ATP regeneration system (20 mM Tris phosphocreatine and creatine kinase). Internal [Cl⁻] or the holding potential were varied, using gluconate as the anion, to alter the direction of Cl⁻ flow, corrected for the junction potential. In some cases, current-voltage curves were constructed using the peak current response to agonist or agonist+THP across a range of holding potentials (−60 to +60) applied as 10 mV steps, or as a voltage ramp. Voltage ramps were generated by ramping the holding potential from −60 to +60 mV in 400 ms in the presence of 1 μM GABA. Traces are presented as the average of 3 traces after subtraction of the leak current. (The leak current was determined in the absence of GABA.) Ramps were performed before and after application of 30 nM THP. Voltage steps were used routinely in addition to voltage ramps to eliminate the possible confound of steroid-induced desensitization¹⁵ which begins to occur within a few ms of GABA application (FIG. 1f). For the experiments examining steroid effects on recombinant GABA receptors, a piezo-controlled double-barreled theta tube (Sutter Instr., 80-100 μm dia.) containing GABA (0.001-1000 μM) or GABA plus THP (30 nM, Steraloids) rapidly (0.3-1 ms onset) delivered drugs to the cell for 400 ms or 2 s exposures (Burleigh Instr., LSS-3100)¹⁴. This rapid application technique yields currents with a rapid rise phase. THP was also bath applied 30 s prior to tests of steroid effects. Currents were recorded using an Axopatch 1D amplifier (Axon Instruments) filtered at 2 kHz (four-pole Bessel filter) and detected at 10 kHz (pClamp 5.1). Analysis of peak current was accomplished with pClamp 9.2 and Origin software (Microcal).

In some cases, saturating concentrations of agonist (100 μM GABA) were rapidly applied (<300 μs onset) for 2 s to assess the rate and extent of desensitization¹⁴ before and after application of THP (via the theta tube). Following the recording, the patch was blown out, and the open tip potential recorded using solutions with a 5% difference in NaCl osmolarity to verify the approximate solution exchange time. The time constant (τ) for desensitization was approximated using non-linear curve fitting routines with Levenburg-Marquardt algorithms (Origin software, Microcal). Goodness of fit was determined by minimizing the sum of the squares of deviations of the theoretical curve from the experimental points.

Unless noted, drugs were from Sigma Chem. Co. Incorporation of the δ subunit was detected by La³⁺ inhibition^{16,17}. In the case of α4β2δ receptors, current amplitudes varied from 75-250 pA consistent with previous reports¹⁸, and much larger than currents seen with α4 β2 (<25 pA). Incorporation of the γ2 subunit was verified by benzodiazepine modulation¹².

Mutagenesis: Site-directed mutations were made using the Quik-Change Mutagenesis Kit I or II (Stratagene Inc., La Jolla, Calif.). Numbering of mutated residues was based on the distance from arginine 99¹⁹. Successful mutations were verified with double-stranded sequencing of end-products (Genewiz Inc., North Brunswick, N.J.).

Hippocampal slice: Slice preparation²⁰: Animals were rapidly decapitated, and the brains removed and cooled using an ice cold solution of artificial cerebrospinal fluid (aCSF) containing (in mM): NaCl 124, KCl 5, CaCl₂ 2, KH₂PO₄ 1.25, MgSO₄ 2, NaHCO₃ 26, and glucose 10, saturated with 95% O₂, 5% CO₂ and buffered to a pH of 7.4. Following sectioning at 400 μm on a Leica oscillating microtome, slices were incubated for one hour in oxygenated aCSF. Slice recording: Pyramidal cells in CA1 hippocampal slice²⁰ or thalamic relay neurons in the ventral tier were visualized using a Leica DIC-infrared upright microscope, and recorded at −50 or −60 mV using whole cell patch clamp procedures at room temperature (20-22° C., Axopatch 200B amplifier, Axon Instruments, 20 kHz sampling frequency, 2 kHz 4-pole Bessel filter) and pClamp 9.2 software. Patch pipets were fabricated to yield open tip resistances of 2-4 MΩ. Internal solution in mM for E_{Cl}=−70 (or E_{Cl}=−30): K-gluconate 141.5 (O), KCl 0 (52.3), CsCl 8.5 (145), HEPES 5, EGTA 5, CaCl₂-H₂O 0.5, QX-314 5 (Calbiochem), Mg-ATP 2, Li-GTP 0.5, pH 7.2, 290 mOsm. (This concentration of QX-314 also blocks GABA_{B} receptors.) When the direction of Cl⁻ current was varied by altering the membrane potential, the internal solution contained cesium-methanesulfonate instead of K-gluconate. (Cesium-methanesulfonate could not be used for the tonic current studies conducted at a −50 mV holding potential because cesium slows, and possibly reverses, the K⁺— Cl⁻ cotransporter KCC²¹, thus compromising efforts to maintain outward Cl current.) The holding potential (Vh) was corrected for the junction potential created by the asymmetric ionic distribution. Electrode capacitance and series resistance were monitored and compensated; access resistance was monitored throughout the experiment, and cells discarded if the access resistance >10 MΩ.

In whole cell recordings of the tonic current, the bath contained 2 mM kynurenic acid and 5-10 mM TEA to pharmacologically isolate the GABAergic current, and in some cases, 200 nM gabazine (6-imino-3-(4-methyoxyphenyl)-1 (6H)-pyridazinebutanoic acid hydrobromide) to selectively block the synaptic GABAergic currents²². In some recordings, 1 μM TTX was added to block action potential-driven GABA release and 1 μM GABA added to record GABA-gated post-synaptic current, in order to rule out potential pre-synaptic effects of the steroid. 30 μM gaboxadol (4,5,6,7-Tetrahydroisoxazolo[5,4-c]pyridin-3-ol, THIP) and/or 300 μM La³⁺ were used to pharmacologically test for the presence of α4 βδ GABA_{A} receptors which have increased efficacy for gaboxadol, and which are uniquely blocked by La³⁺, compared to other GABA_{A} receptor isoforms^{16,17}. In some cases, 50 μM L-655,708 was added to the bath solution to block α5-containing GABA_{A} receptors²³.

Tonic current was recorded as the difference current produced by 120 μM gabazine, which blocks all GABA_{A} receptor, before and after 30 nM THP²⁴. Current was also recorded in response to 2 s 10 mV steps in the holding potential, and current-voltage curves generated to the steady-state current. Because fast-inactivating conductances would not be excluded in a voltage ramp, stable steady-state current responses were used instead to construct the current-voltage (IV) curves. Input resistance (Rm) was calculated from the current response to voltage steps from −60 to −40 mV using Ohm's Law. The reversal potential was determined by the y intercept of the IV curve (−90 to −10 mV). The gabazine-sensitive slope conductance was assessed as the slope of the linear portion of outward current plot recorded before and after THP after subtraction of the gabazine-generated current (in the presence of L-655,708 to block α5-GABA_{A} receptors). Only data with a stable baseline and pipet access resistance <10 MΩ were included in the analysis.

Recombinant receptors: Human embryonic kidney (HEK)-293 cells were transfected with α4 β2δ or other indicated subunit combinations (see Supplementary Methods) and co-transfected with enhanced green fluorescent protein for visualization. GABA-gated currents were recorded at room temperature (20-22° C.) at a holding potential of −50 mV¹⁷ using a pipet solution containing 120 mM: N-methyl-D-glucamine chloride. Internal [Cl⁻] or the holding potential were varied to alter the direction of Cl⁻ flow. A piezo-controlled double-barreled theta tube (Sutter Instr., 80-100 μm dia.) containing GABA (0.001-1000 μM) or GABA plus THP (30 nM, Steraloids) delivered drugs to the cell for 400 ms or 2 s exposures (see next section). In some cases, current-voltage curves were constructed using the peak current response to agonist or agonist+THP across a range of holding potentials (−60 to +60) applied as 10 mV steps, or as a voltage ramp generated by ramping the membrane potential from −60 to +60 mV (over 400 ms) in the presence of 1 μM GABA. Ramps are presented as the average of 3 traces after subtraction of the leak current (obtained in the absence of GABA). Currents were recorded using an Axopatch 1D amplifier (Axon Instruments) filtered at 2 kHz (four-pole Bessel filter), detected at 10 kHz and analyzed with pClamp 9.2. Desensitization rate was determined using non-linear curve-fitting routines (Origin, Microcal; see next section).

Further Studies with Recombinant receptors: Transfection—Plasmids obtained from S. Vicini (rat α1, α5, β2, β3 and y2), P. Whiting (human α4 and 6) and N. L. Harrison (mouse α4) were propagated in E coli DH5α and prepared using Qiagen Maxi- or Midi-prep kits. (Rat, mouse and human α1, β2, β3 and γ2 subunits are identical or nearly identical.) All receptors were transfected at a 1:1:1 ratio, except a132γ2 (1:1:5)¹², α4β28 (5:1:5)¹³ and α4β2γ2 (5:1:1). HEK-293 cells were maintained in medium (DMEM:Ham's F-12 1:1) supplemented with 10% fetal calf serum at 37° C. in a humid 5% CO₂ atmosphere. Cells were transfected using calcium phosphate or Polyfect (Qiagen) and co-transfected with enhanced green fluorescent protein (ratio of DNA to eGFP was 6:1 or 10:1 depending on the DNA concentration) for visualization. a4-containing GABA_{A} receptors required 2-3 d for maximal levels of expression; all other receptors were recorded 1-2 d after transfection. Because mouse α4 and human a4-containing receptors yielded the same results, the currents were averaged together.

Hippocampal slice (see Supplementary Methods for more detail): Pyramidal cells in CA1 hippocampal slice (400 μm) or thalamic relay neurons were visualized with DIC-microscopy and recorded at −50 or −60 mV at room temperature (20-22° C.) using whole cell patch clamp procedures (Axopatch 200B amplifier, Axon Instruments, 20 kHz sampling frequency, 2 kHz 4-pole Bessel filter) and pClamp 9.2 software. The direction of Cl⁻ current was varied by altering internal Cl⁻ (K-gluconate and KCl, internal solution) or by applying 10 mV voltage steps (−90 to −10 mV, 2 s). Kynurenic acid (2 mM) and TEA (5 mM) were added to the bath solution to isolate the GABAergic current, and 200 nM gabazine to isolate the non-synaptic GABAergic current³⁰. Action potential-driven GABA release was blocked with 1 μM TTX, and 1 μM GABA added to generate post-synaptic GABA-gated current.

Tonic current was recorded as the difference current produced by the selective GABA_{A} receptor antagonist gabazine (120 μM) before and after 30 nM THP^{4,29}, while gramicidin perforated-patch recordings³³ were accomplished using 140 mM KCl plus 25 μg ml⁻¹ gramicidin in the pipet solution, recorded when the access resistance dropped to <60 MΩ after tight seal formation. Estimates of the direction of Cl⁻ current were obtained by recording using tight-seal cell-attached techniques³¹ (>1 GΩ seal) in current clamp mode. A downward deflection signified outward (i.e., hyperpolarizing) Cl⁻ current.

Effects of THP on cell excitability were tested by monitoring spiking using cell attached patch recordings³¹ in voltage clamp mode (−40 mV holding potential, 150 mM NaCl intrapipet solution) or assessing the current threshold to spiking and spike frequency in current clamp mode (0.01-0.3 nA steps, starting from −1 nA, 1 s duration). (More complete details are supplied in the Supplementary Methods.)

Restraint stress. In order to test the effect stress-induced release of THP^{6,46,47} on anxiety, mice were restrained in a clear Plexiglas tube-type holder (Harvard Apparatus) for 45 min. and tested 20 min. later on the elevated plus maze (see Supplementary Methods). Open arm time was evaluated for 5 min. on the elevated plus maze. A decrease in open arm time reflects an increase in anxiety¹⁸. In all cases, the results from each mouse tested after restraint was expressed relative to the averaged results from the sham controls, which were identical to the stressed animals (age, genotype, sex, drug-injected), except that they were not subjected to restraint stress.

Statistics All data are presented as mean±SEM. Complete details on the statistical procedures are provided in the Supplementary Material. Comparisons of pre- and post-THP values of the GABA-gated current recorded from the same cell were determined using the paired t-test. Comparisons between >2 groups were assessed using an analysis of variance (ANOVA) following confirmation that the data followed a normal distribution with the Kolmogorov-Smirnov normality test. Unless otherwise noted, statistical significance was achieved when P<0.05.

### Example 2

### Results

Effects of THP on α4β2δ GABA_{A} receptors

In contrast to its effect at other receptor subtypes, 30 nM THP decreased the outward GABA (1 μM)-gated Cl⁻ current through recombinant α4 β2δ receptors expressed in HEK-293 cells by 28±3% (mean±SEM, FIG. 1,b, FIGS. 11a,b, P<0.05, statistics summarized in the Supplementary Material, Table 1), recorded at −50 mV with whole cell patch clamp techniques. When assessed across a range of voltage steps (FIG. 1c, FIG. 12), THP significantly decreased the conductance of the outward current by 36-43%. This action of the steroid was not directly influenced by the membrane potential (FIG. 1c). Thus, in experiments where we varied the reversal potential for Cl⁻ by altering internal Cl⁻ concentration, THP produced equivalent decreases in outward current at a similar Cl⁻ driving force when assessed at different membrane potentials. However, THP application did not itself alter the Cl⁻ reversal potential (FIGS. 1c,d, FIG. 12) suggesting that it does not alter non-GABA-gated conductances. Similar decreases in outward current were produced by THP assessed using a voltage ramp (FIG. 1d). In contrast, THP robustly increased inward currents through these receptors (FIGS. 1a,b). The concentration of GABA used here (1 μM) is an EC₇₅ for α4 β2δ GABA_{A} receptors (FIG. 11, and represents the GABA concentration to which extrasynaptic GABA_{A} receptors, such as α4β6, would be exposed²⁴. In contrast, 30 nM THP applied without GABA had no effect (data not shown). We also studied various receptor subtypes using an EC₂₀ concentration of GABA (5-10 μM for most receptors, FIG. 1b). In contrast to its effects at α4β2δ, THP either increased or had no effect on the outward current at α1β2δ, α4β3δ, α1β2γ2, α4β2γ2 and α5β2/3γ2 receptors (FIGS. 1a,b). Thus, the inhibitory effect of THP is dependent on the presence of α4, β2 and δ subunits, and was selective for the active 3α-OH isomer, but not the inactive 3,3-OH isomer⁵, of THP (FIG. 1e).

One potential mechanism for the THP-induced decrease in outward current at α4β326 GABA_{A} receptors is through acceleration of receptor desensitization¹¹. Therefore, we used rapid application techniques to administer saturating concentrations of GABA (100 μM) for 2 s to HEK-293 cells expressing α4 β2δ GABA_{A} receptors. In fact, 30 nM THP increased desensitization of outward currents from 8±2% to 87±5.6% (100 μM GABA, P<0.001, FIG. 1f), with a markedly faster time-course (τ=230±35 ms versus pre-THP, 1700±200 ms, P<0.001). Although peak current was unchanged by steroid exposure, the amplitude of the desensitized current <50 ms after application of GABA was significantly smaller than control. This desensitized state is relevant for tonic current which is equivalent to the steady-state current. Consistent with this, the decrease in outward steady-state current was correlated with GABA concentration, with THP producing a greater decrease in current gated by higher concentrations of GABA where desensitization is more pronounced (FIGS. 1a,f; FIGS. 11,12)

### Residues Required for THP Inhibition of α4βδ Receptors

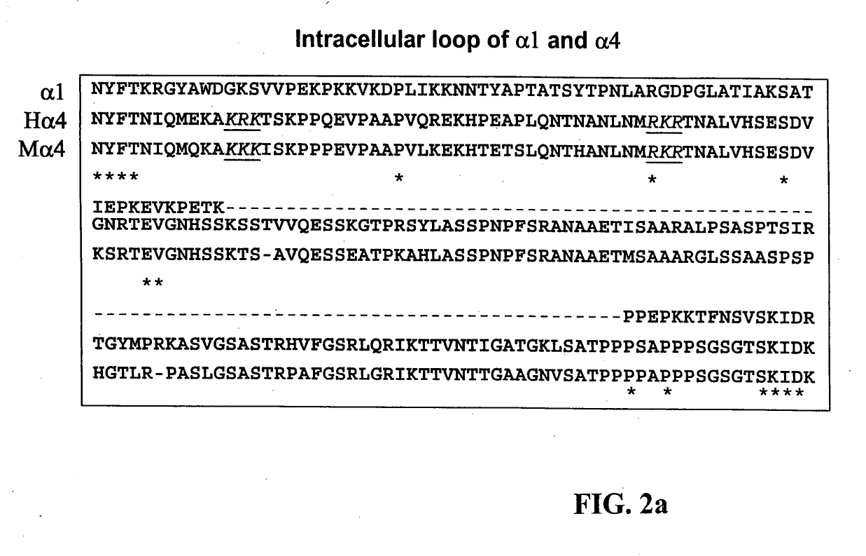

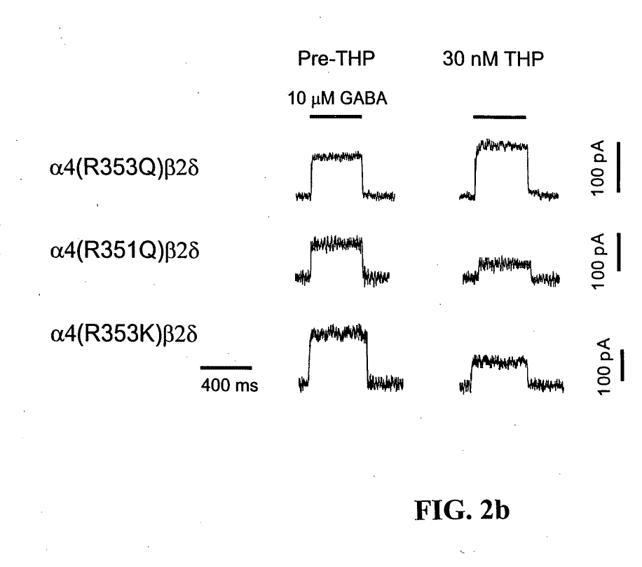

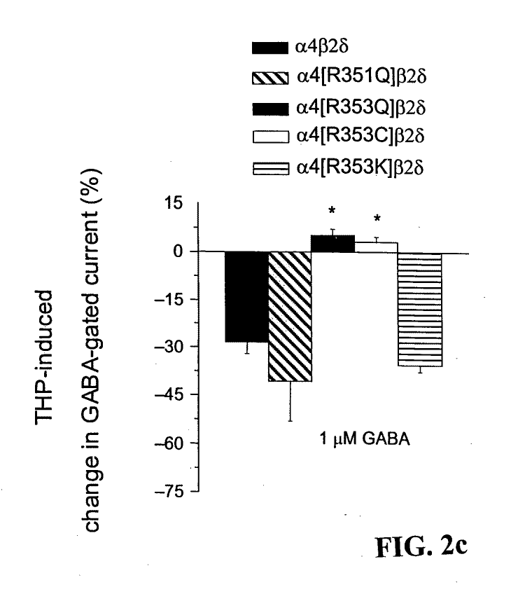

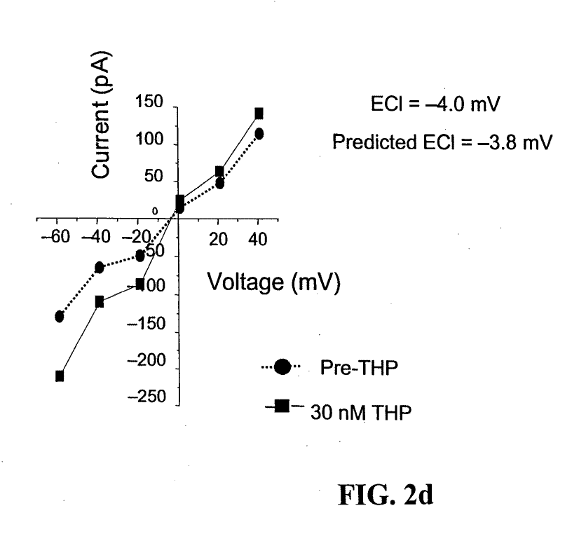

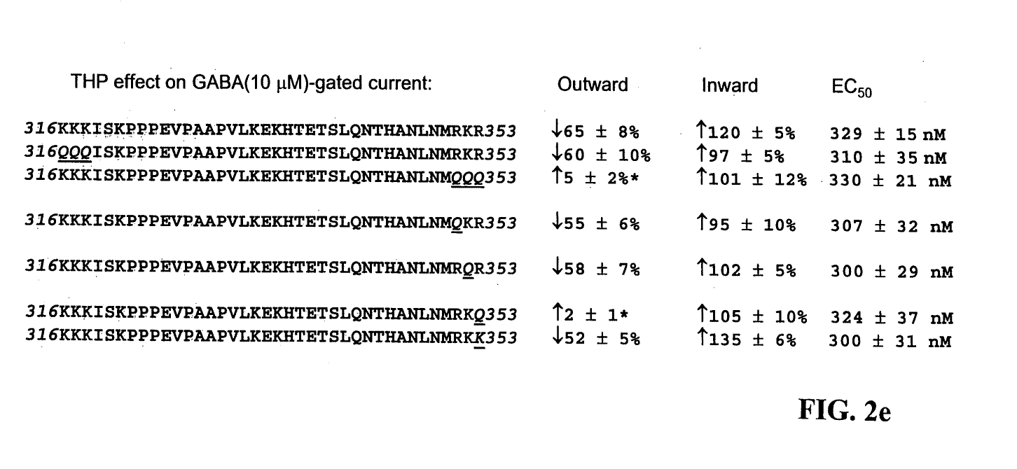

The α1 and α4 subunits have the least homology in the intracellular loop region (FIG. 2a), which may contribute to the permeation pathway in the Cys-loop family of receptors^{25,26}. Because recent studies have reported the existence of charged residues which are ion sensor sites in membrane proteins²⁵⁻²⁷, we investigated whether positively charged residues within the loop might mediate the Cl⁻ dependent effects of THP seen at α4 β2δ receptors. Indeed, mutation of a positively charged arginine (R) at position 353 to a neutral glutamine (Q) or cysteine (C) residue in the α4 subunit prevented the steroid-induced reduction in outward current of α4 β2δ GABA_{A} receptors expressed in HEK-293 cells (FIG. 2b-e, Supplementary Tables 2-4), whereas mutation of R353 to another basic residue, lysine (K), did not prevent THP inhibition of outward current (FIGS. 2b,c,e).

Mutations at nearby arginine or lysine residues R351Q, K352Q, K316Q, R317Q and K318Q had no effect (FIGS. 2b,d,e) suggesting that residue 353 was uniquely involved in steroid inhibition of the outward current. In contrast, THP increased inward current through α4[R353Q]β2δ GABA_{A} receptors, and this mutation did not alter sensitivity to GABA or the ECl, determined before and after THP administration (FIGS. 2d,e). These results suggest that a basic residue at position 353, a putative Cl⁻ modulatory site, is necessary and sufficient for Cl⁻ dependent THP inhibition of α4 β2δ GABA_{A} receptors.

### Localization of α4 and δ Subunits in CA1 Hippocampus

α4 βδ receptors are normally expressed at very low levels in CA1 hippocampal pyramidal cells¹⁶. Given the novel effects of THP at these receptors, we hypothesized that their expression may be altered during puberty when the anxiety response to stress is increased³. Initially, we localized α4 and δ subunits in CA1 hippocampus using immunohistochemical techniques at the onset of puberty in female mice, defined as the first metestrus stage after vaginal opening. Markedly increased expression of α4 and δ was observed along the pyramidal cell dendrites in the stratum radiatum of CA1 hippocampus at puberty (FIGS. 3a,b) from almost undetectable levels before puberty, as reported in the adult¹⁶. In fact, expression of both α4 and δ subunits was increased by up to two-fold (P<0.05) at the onset of puberty (FIGS. 3c,d, Table 5), quantified using Western blot techniques.

### Puberty and Hippocampal THP Levels

In addition to upregulation at the onset of puberty, expression of α4 and δ subunits increases in adult hippocampus when circulating levels of THP decrease (i.e., “THP withdrawal”)^{17,19}. Thus, we determined whether endogenous THP levels decrease across pubertal development. In fact, hippocampal THP levels declined by 56±12% (P<0.05, n=8) at the onset of puberty, as has been shown previously for humans when fluctuating levels of THP follow prolonged elevations of the steroid prior to puberty onset²⁸.

The decline in THP levels we observe in mouse hippocampus was similar to that produced by administration of a 5α-reductase blocker (58±10%) which prevents formation of THP¹⁸. Accordingly, increases in α4 and δ expression were also seen following THP withdrawal (FIGS. 3c,d). Because increased expression of α4 and 6 subunits at the onset of puberty was prevented by replacement THP (10 mg kg⁻¹ day⁻¹ for 3 days, FIGS. 3c,d), these results suggest that declining levels of THP at puberty trigger expression of α4 and δ subunits. In contrast to α4, expression of the α5 subunit, which underlies most tonic inhibition in the CA1 hippocampus²⁹, was unchanged by puberty (data not shown).

### THP and Tonic Current

GABA_{A} receptors containing α4 and δ subunits are localized to extrasynaptic sites¹³ where they generate a tonic current responsive to low concentrations of steroid⁴. Therefore, we reasoned that THP would reduce the outward tonic GABAergic current after puberty, when α4 βδ receptors are expressed at high levels, an effect verified through selective pharmacological tests (FIG. 13, Table 6) in addition to immunocytochemical and Western blot detection (FIG. 3). Indeed, in hippocampal slices from pubertal mice, 30 nM THP reduced the tonic current (FIGS. 4a,b) by 48±6%, recorded with whole cell patch clamp techniques from CA1 pyramidal cells using low internal Cl⁻ to achieve outward current. Based on our findings with recombinant receptors, we also predicted that the inhibitory effect of THP on tonic GABAergic currents would be prevented if the direction of the Cl⁻ current were reversed. Indeed, THP increased the tonic GABAergic current when the cell was loaded with Cl⁻ to produce inward current (FIG. 4a inset, 4b). In these recordings, the synaptic current was selectively blocked with a low concentration of gabazine (200 nM), a GABA_{A} receptor antagonist³⁰, to visualize the tonic current.

In contrast, THP increased the outward tonic current before puberty and in the δ^{-/-}mouse after puberty (FIGS. 4a,b), both conditions where α4 βδ GABA_{A} receptors have low levels of expression. Interestingly, THP produced similar decreases in outward current after THP withdrawal (FIG. 4b) suggesting that the decline in THP at puberty results in this paradoxical inhibitory effect of the steroid on outward tonic current. In contrast to the steroid-induced decrease in tonic current, baseline levels of tonic current were increased at puberty (FIG. 4a), however, compared to levels in pre-pubertal slices.

### Cell-Attached and Perforated-Patch Recordings

To determine whether THP inhibition of GABAergic currents at puberty was a physiological phenomenon, we initially verified that GABA-gated currents were outward in CA1 hippocampal pyramidal cell dendrites at the onset of puberty. To this end, we recorded the change in membrane potential produced by local application of the GABA agonist gaboxadol (4,5,6,7-Tetrahydroisoxazolo[5,4-c]pyridin-3-ol, THIP) to the apical dendrite in the stratum radiatum using the hippocampal slice preparation. The voltage change was recorded in current clamp mode from the soma using tight-seal cell attached techniques at a holding current of 0 pA³¹. A downward shift of the membrane potential produced by dendritic gaboxadol application indeed verified hyperpolarizing GABAergic dendritic current (FIG. 4c), as suggested by other reports^{22,23}.

One complication of whole-cell patch clamp recording is that normal ionic gradients are disrupted. Therefore, in order to verify that THP reduced tonic GABAergic currents in intact cells under conditions of unperturbed internal Cl⁻, we directly recorded pharmacologically isolated tonic GABAergic current from the soma using perforated-patch voltage-clamp techniques in the hippocampal slice. In order to rule out potential pre-synaptic effects, 1 μM tetrodotoxin (TTX) was used to block activity-driven GABA release, and instead post-synaptic current was generated with the addition of 1 μM GABA added to the bath solution. Under these conditions, 30 nM THP produced a downward shift in the holding current (FIGS. 4d,e), similar to the GABA antagonist gabazine (120 μM). Thus, THP effectively depressed the outward GABAergic current by 40±8% in slices from pubertal animals. THP also decreased the GABA-gated conductance (FIGS. 5a,b), assessed as the slope of the gabazine-sensitive current in response to 10 mV steps (−60 to −40 mV). However, THP did not alter the reversal potential (FIG. 5a), suggesting that it did not alter conductances of other channels. Distinct from its effect at puberty, THP had no effect on the post-synaptic GABAergic tonic current (FIGS. 4d,e) before puberty when α4 βδ expression is low (FIG. 3). Interestingly, THP also reduced the tonic current in pre-pubertal thalamic relay neurons by 57±12% (P<0.05, n=6, data not shown), which normally have high levels of α4β2δ expression^{16,32} that underlie a tonic current³², where GABA-gated current is outward³³. Thus, α4 β2δ GABA_{A} receptor expression and outward Cl⁻ current are necessary and sufficient for the paradoxical effect of THP.

### THP and Neuronal Excitability

We reasoned that the decrease in the tonic dendritic GABAergic conductance produced by THP at puberty would increase input resistance. Indeed, THP significantly increased the input resistance by 38±5%, calculated from the current response to 10 mV steps (−60 to −40) in the hippocampal slice. (FIG. 14, Tables 7,8). This effect was not seen in slices from δ^{-/-}mice, and was prevented when 120 μM gabazine was pre-applied, demonstrating that alterations in the GABA-gated conductance underlie the change.

Increases in the input resistance produced by THP would be predicted to increase neuronal excitability at puberty. Indeed, THP significantly (P<0.001) increased spiking at this time, assessed in cell-attached mode³¹ where the internal Cl⁻ was undisturbed (FIGS. 5c,d, Tables 9,10). Baseline levels of neuronal excitability were reduced at puberty, however, as expected for an increase in tonic current. In order to determine the cellular characteristics which might underlie this event, we also conducted whole cell recordings (FIGS. 6a,b) in current clamp mode where we monitored spiking of CA1 hippocampal pyramidal cells in response to progressively increasing levels of injected current. Here, THP reduced the amount of current necessary for triggering a spike (I threshold, FIGS. 6a,b) at puberty. THP also increased action potential frequency in these cells (FIGS. 6a,b), without changing spiking characteristics or other membrane properties such as voltage threshold, action potential amplitude or action potential half-width (FIG. 6b, Tables 11,12). Although the onset of puberty was also associated with a “sag” in the voltage response to hyperpolarizing current injection, suggesting the presence of Ih (hyperpolarizing-induced cation current), selective blockade of this current with 20 μM ZD 7288 did not prevent the excitatory effect of THP on CA1 hippocampal pyramidal cells (FIGS. 6a,b). Blockade of Ih altered the after-hyperpolarization to more closely approximate its pre-pubertal level, also ruling out changes in after-hyperpolarization as a potential mechanism for the effect of THP at puberty. This excitatory effect of THP on neuronal firing was not observed in hippocampal slices from δ^{-/-} mice, implicating 6-containing receptors. In contrast, before puberty, THP decreased neuronal excitability (FIGS. 5c,d; 6a,b), evidenced by a decrease in the current threshold for spiking and reduced spike frequency at threshold.

### THP, Stress and Anxiety Behavior

Consistent with the in vitro findings, the onset of puberty reversed the behavioral effect of THP from decreasing anxiety, as normally observed⁸, to increasing anxiety (FIG. 7a, Tables 13,14). To study this, we used an animal model in which the time spent on the open arm of an elevated plus maze reflects a decrease in anxiety¹⁸. In fact, following the onset of puberty, acute administration of THP at a physiological dose (10 mg kg⁻¹, intraperitoneally) decreased open arm time by 35±8% on the elevated plus maze, without changing locomotor activity. We have reported similar paradoxical anxiety-producing effects of THP after THP withdrawals, when α4 βδ GABA_{A} receptors are increased. Because endogenous THP is released by stress^{6,34}, we also tested this physiological outcome by assessing anxiety behavior 20 min after restraint stress. As predicted by the anxiogenic effect of THP, restraint stress also significantly increased anxiety in pubertal mice (decreasing open arm time by 27±2.6%, P<0.05), in contrast to its anxiety-reducing effect in pre-pubertal mice and in adult mice (FIG. 7a).

Stress is also associated with activation of the hypothalamo-pituitary-adrenal axis³⁵. Therefore, we verified that effects of restraint stress were due to THP by pre-administration of the inactive 3β-OH isomer, an antagonist of THP effects at GABA_{A} receptors³⁶ (FIG. 1b), and blockade of endogenous THP formation, which both prevented the stress-induced increase in anxiety (FIG. 7a). Stress-related increases in anxiety after puberty were not observed in δ mice, implicating 6-containing receptors. Replacement THP was also administered at puberty to prevent the decline in THP. Animals tested after this steroid replacement paradigm did not exhibit an anxiety response to stress, suggesting that the decline in THP underlies the anxiety-producing effect of stress. In contrast, anxiety level was not different among the various developmental and treatment groups not exposed to stress, reflected by the open arm time (FIG. 7b), nor was locomotor activity (mean change=1.6±3%).

Results demonstrate that effects of the neurosteroid THP can reverse from its classic effect of enhancing GABA-gated current to inhibiting current at α4 β2δ GABA_{A} receptors in a Cl⁻ dependent manner. Expression of these receptors was increased in CA1 hippocampus at the onset of puberty, where they generated an outward current. Under these conditions, THP paradoxically increased anxiety in contrast to its well-known anxiety-reducing effect in pre-pubertal and adult animals⁸.

The inhibitory effect of THP on outward currents at α4 β2δ GABA_{A} receptors was dependent upon arginine 353 in the intracellular loop of α4, a basic residue that may act as a modulatory site for Cl⁻ Recent studies suggest that ion sensor sites can regulate other events such as Cl⁻ activation of HCN subunits which mediate the hyperpolarization-activated cation current (Iₕ)²⁷. In addition, the recent discovery of a cation-triggered phosphorylation event in a novel membrane protein lacking an ion pore³⁷ suggests that ion sensor sites regulate neuronal function beyond ion conductance. Modulatory effects of Cl⁻ have been noted before³⁸, which are necessary for barbiturate and benzodiazepine binding. In addition, the intracellular loop of the Cys-loop family of receptors is ion accessible^{25,26}, while for other membrane receptors this loop functions not only as a permeation pathway, but also as a site necessary for rapid desensitization. Indeed, the effect of THP was to promote rapid desensitization of the receptor, an effect leading to reduced current amplitude. Direction-sensitive changes in the rate of desensitization have been reported for GABA_{A} receptors, including the homologous α6β3δ¹¹, at which the outward Cl⁻ current desensitizes more then the inward current. Our data are also consistent with the finding that neurosteroids facilitate desensitization of δ-containing GABA_{A} receptors⁴⁰, but are novel in demonstrating effects of low nanomolar concentrations of THP at an ambient concentration of 1 μM GABA, relevant for the physiological state²⁴.

α4 and δ subunits are localized extrasynaptically¹³ where they co-express with β2³². These α4 β2δ GABA_{A} receptors have a high sensitivity¹⁹ to low concentrations of GABA and a relative lack⁴⁰ of desensitization making them ideally suited to generate a tonic current. However, by increasing receptor desensitization of α4βδ GABA_{A} receptors at puberty, THP reduced this tonic inhibition of CA1 hippocampal pyramidal cells. This reduction in conductance along the dendrites increased the input resistance of the neuron, similar to effects reported after blockade of dendritic K⁺ channels⁴¹. Increasing the input resistance would allow ongoing excitatory synaptic currents to produce a larger depolarizing effect on the cell body of the neuron, thus increasing the likelihood of triggering an action potential. Alterations in this type of shunting inhibition have been shown to affect both sub-threshold events, as well as drive a higher firing frequency⁴², consistent with the results shown here. In contrast, action potential characteristics were not altered nor was the voltage threshold for triggering an action potential, suggesting that changes in excitatory transmission were not affected by THP. Other conductances, such as Ih and K⁺ channel current, were similarly not involved in the excitatory effects of THP, which were solely dependent on the presence of 6-containing GABA_{A} receptors.

The results presented herein indicate that the effects of THP predominate at the output neurons of the hippocampus at puberty because application of THP reduced tonic inhibition generated either by ambient GABA or following addition of GABA to the slice while blocking interneuron activity with TTX. This effectively led to increases in excitability of CA1 pyramidal cells. Increases in excitability of the major output neurons of the hippocampus produced by THP would impact upon behavioral end-points influenced by this limbic structure⁸, leading to increased emotional reactivity, which we observed. In fact, recent evidence⁴³ suggests that anxiety-reducing effect of benzodiazepines is due to direct modulation of the tonic current, as has also been shown for the anti-seizure effect of the GABA agonist gaboxadol⁴⁴.

In contrast to its effect at puberty, THP had no effect on the post-synaptic tonic current recorded from CA1 pyramidal cells before puberty when expression of α4 βδ receptors is low¹⁶. This is consistent with the finding that the extrasynaptic receptors present at this time, which contain the α5 subunit²⁹, are relatively insensitive to THP¹². In contrast, α4 β2δ receptors are expressed at high levels on the dendrites of dentate gyrus granule cells¹⁶, where the GABAergic current is inward²⁰. Thus, THP and related steroids enhance inhibition of this limbic structure⁴, consistent with their anxiety-reducing effect before puberty and in the adult⁸.

The anxiety-promoting effect of THP at the onset of puberty may contribute to the aversive effects of stress which emerge at puberty in humans³. Distinct from effects of corticosterone, which are long-lasting⁴⁵, release of THP is a relatively short-term response to acute stress, with a time-course of one to two hours after the event, as demonstrated in both rodents⁴⁶ and humans³⁴, when decreases in anxiety occur⁶. The THDOC metabolite of corticosterone (5α-pregnane-3α, 21-diol-20-one) is also released following stress, but at a lower concentration^{46,47} across a shorter time-frame than THP⁴⁶. As a similar neuroactive steroid, it would likely contribute to the effect exerted by THP. In contrast, baseline levels of anxiety were not altered by puberty in female mice. Instead, the stress-induced increase in anxiety produced by THP in adolescent females would be evidenced as a transient increase in anxiety, reflected as a “mood swing”. Emotional changes also occur in males, but these may additionally involve changes in male-specific steroids⁴⁵ which can also alter mood.

Steroid fluctuations in the adult also result in anxiety-producing effects of THP: These include premenstrual syndrome^{48,49} and post-menopausal irritability⁵⁰. Taken together, these results suggest that a reversal of the normally anxiety-reducing effect of THP via effects at α4β2δ GABA_{A} receptors may represent an adaptive response to steroid fluctuations when increases in emotional reactivity occur.

### Example 3

### Behavioral Studies

Elevated plus maze. Testing rodent behavior on the elevated plus maze is an established animal model of anxiety³¹. The plus maze consists of four 8×35 cm arms at 90° angles, elevated 57 cm above the floor. Two arms are enclosed by 33 cm walls, and two arms have no walls (“open arms”). The open arms are also partially bordered by small rails (5×15 cm) extending to the proximal half of the arm, and the floor of the maze is marked with grid lines every 25 cm. Each animal was initially acclimated to the room for 30 min-1 h before being placed in the center of the maze, and exploratory activity recorded for 5 min. In some cases, animals were exposed to restraint stress (see below) 20 min before plus maze testing. Background white noise was used for all tests. The time spent in the open and closed arms was tabulated, as were the entries. To be considered an open arm entry, the animal had to cross the line of the open platform with all four paws. An increase in time spent in the open arm is considered to be a measure of decreased anxiety³¹, as we have described³². The number of total entries is a measure of general activity level.

Restraint stress. In order to test the effect stress-induced release of THP⁵ on anxiety, mice were restrained in a clear Plexiglas tube-type holder (Harvard Apparatus) for 45 min. and tested 20 min. later on the elevated plus maze (see above). Robust 20 to 100-fold increases in brain THP levels have been reported after restraint stress up to 120 min after the stress³³⁻³⁵. In all cases, the results from each mouse tested after restraint was expressed relative to the mean value for a sham control group, identical to the stressed mouse in all respects (age- and genotype-matched), but not exposed to restraint.

Initially, all data was shown to fit a normal distribution using the Kolmogorov-Smirnov test for normality. Planned comparisons for changes in GABA-gated current before and after THP treatment for the same cell were analyzed using a paired t-test (recombinant and hippocampal slice studies)³⁶. For the perforated patch recordings, comparisons of THP effects between pre-pubertal and pubertal groups were performed with a Students t-test. Comparisons of THP-induced changes in current between multiple groups were analyzed with an analysis of variance (ANOVA), as were comparisons of the GABA EC₅₀ between different recombinant GABA_{A} receptor subtypes. Post-hoc comparisons for the ANOVA were made with a Tukey's or a Student-Newman-Keuls post-hoc test. Current clamp experiments, Because multiple comparisons were made on a single cell, a two-way ANOVA was used to compare differences in the various parameters between multiple groups, followed by a post-hoc Holm-Sidak test. For all tests, the levels of significance was determined to be P<0.05, unless otherwise noted. Sigma-Stat 3.5 and Statistica were used to perform the comparisons with assistance from Zar³⁶. F values are presented below for all experiments. In addition, selected post-hoc comparisons are indicated for experiments with >3 groups.

Behavioral experiments: Initially, the data was shown to fit a normal distribution using the Kolmogorv-Smirnov normality test. Then stress-effects on mice with different pubertal and/or drug-treatment status were compared using an ANOVA. The post-hoc test chosen for this was the Least Significant Difference comparison between either the pre-pubertal or pubertal groups and all other groups. This test is recommended for planned comparisons³⁶, where significant differences can be determined independently of differences in n or the magnitude of the effect. An additional post-hoc comparison, Duncan's test, was also implemented, and revealed similar statistical comparisons. Here we present comparisons between percent changes in open arm time, but similar statistical outcomes were obtained when the absolute values of open arm time were compared across groups. For all tests, the levels of significance was determined to be P<0.05, unless otherwise noted. (Statistica was used to perform the comparisons.) F values and planned comparisons are indicated in the tables below.

### Tables of F Values

Each table below indicates the group degrees of freedom (DF), the residual DF and F values, as well as the critical value for F or P value. Significant F values are indicated with an asterisk for each ANOVA comparison. Selected planned comparisons are also presented where >3 groups are compared.

TABLE 1
ANOVA - F Tables - RECOMBINANT RECEPTOR EXPERIMENTS

| | | Group | | | |
|---|---|---|---|---|---|
| FIG. 1b | | DF | Res DF | F | Significant F |
| Upper | Cl⁻ out | 3 | 21 | 48* | 8.99 |
| panel | | | | | (p = 0.001) |
| | Cl⁻ in | 3 | 21 | 6.7* | 3.95 |
| | | | | | (p = 0.05) |
| Lower | Cl⁻ out | 3 | 21 | 44* | 8.99 |
| panel | | | | | (p = 0.001) |
| | Cl⁻ in | 3 | 21 | 6.8* | 3.95 |
| | | | | | (p = 0.05) |
| FIG. 1c | | 3 | 18 | 0.08 | 3.95 |
| gSlope | | | | | (p = 0.05) |

TABLE 2
MUTATIONS

| FIG. 2 | | Group | | | |
|---|---|---|---|---|---|
| c, e | | DF | Res DF | F | Significant F |
| 1 μM | Cl⁻ out | 4 | 21 | 12.55* | 7.83 |
| GABA | | | | | (p = 0.001) |
| 10 μM | Cl⁻ out | 6 | 29 | 21.70* | 5.73 |
| GABA | | | | | (p = 0.001) |
| | Cl⁻ in | 6 | 28 | 2.90 | 2.90 |
| | | | | | (p = 0.05) |
| EC₅₀ | | 6 | 28 | 0.20 | 2.90 |
| | | | | | (p = 0.05) |

TABLE 3
Tukey's test - planned post-hoc comparisons
Mutations - 1 μM GABA

| Group | Comparison | P |
|---|---|---|
| A4β2δ | α4(R353Q)β2δ | .002* |
| | α4(R353C)β2δ | .003* |
| A4(R353Q)β2δ | α4(R351Q)β2δ | <0.001* |
| | α4(R353K)β2δ | .002* |
| A4(R353C)β2δ | α4(R351Q)β2δ | <0.001* |

TABLE 4
Mutations - 10 μM GABA

| Group | Comparison | P |
|---|---|---|
| A4β2δ | α4(RKR351- | <0.001* |
| | 353QQQ)β2δ | |
| | α4(R353Q)β2δ | <0.001* |
| α4(RKR351- | α4(KKK316- | <0.001* |
| 353QQQ)β2δ | 318QQQ)β2δ | |
| A4(R353Q)β2δ | α4(R351Q)β2δ | <0.001* |
| | α4(R352Q)β2δ | <0.001* |
| | α4(R353K)β2δ | <0.001* |

TABLE 5
WESTERN BLOT

| | Group | | | |
|---|---|---|---|---|
| FIG. 3d | DF | Res DF | F | Significant F |
| α₄ | 3 | 20 | 4.4* | 3.86 |
| | | | | (p = 0.001) |
| δ | 3 | 20 | 4.4* | 3.86 |
| | | | | (p = 0.001) |

TABLE 6
SLICE PHARMACOLOGY

| Supp. | Group | | | |
|---|---|---|---|---|
| FIG. 3 | DF | Res DF | F | Significant F |
| THIP | 3 | 18 | 27.6* | 9.69 |
| THIP/LA | 3 | 18 | 11.8* | 9.69 |
| Difference | | | | |

TABLE 7
SLICE PHYSIOLOGY

| Supp. FIG. 4 | Group DF | Res DF | F | P |
|---|---|---|---|---|
| Input | 5 | 44 | 33 | <0.001* |
| resistance | | | | |

TABLE 8
Tukey's post-hoc comparisons

| Group | Comparison | P |
|---|---|---|
| Pre-pubertal | Pubertal | <0.001* |
| | THP Wd | <0.001* |
| Pubertal | Pub + gabazine | .008* |
| | Pub δ^{-/-} | <0.001* |
| | Pub + repl THP | <0.001* |

TABLE 9
Neuronal excitability

| | Group | | | |
|---|---|---|---|---|
| FIG. 5 | DF | Res DF | F | Significant F |
| gslope | 2 | 14 | 4.1* | 3.68 |
| after | | | | (0.05) |
| THP | | | | |
| Spiking | 5 | 24 | 40.4* | 6.68 |
| (cell- | | | | (0.001) |
| attached) | | | | |

TABLE 10
Cell-attached spiking - Tukey's test - planned post-hoc comparisons

| Group | Comparison | P |
|---|---|---|
| Pre-pubertal | Pubertal | <0.001* |
| | THP Wd | <0.001* |
| Pubertal | Pub δ^{-/-} | <0.001* |
| | Pub + repl THP | <0.001* |
| THP Wd | THP Wd δ^{-/-} | <0.001* |

TABLE 11
Two-Way ANOVA

| Current Clamp | | | | | |
|---|---|---|---|---|---|
| Data - FIG. 6 | DF | Res DF | Total DF | F | P |
| GroupxParameter | 12 | 114 | 133 | 8.315* | <0.001 |

TABLE 12
Holm-Sidak post-hoc comparison

| Current clamp | | | | |
|---|---|---|---|---|
| data - FIG. 6 | Groups | t | P | Critical level |
| Current | Pre vs. Pub | 7.911 | <0.0001* | .009 |
| threshold | Pub vs. | 1.378 | 0.171 | 0.05 |
| | Pub-ZD | | | |
| | Pub vs. | 2.661 | .009* | .025 |
| | Pub δ^{-/-} | | | |
| Vm | Pre vs. Pub | .0482 | .962 | 0.017 |
| threshold | Pub vs. | .0821 | .935 | .01 |
| | Pub-ZD | | | |
| | Pub vs. | .00614 | .995 | .05 |
| | Pub δ^{-/-} | | | |
| Spike | Pre vs. Pub | 5.038 | <0.0001* | .009 |
| frequency | Pub vs. | 2.523 | .013 | .013 |
| | Pub-ZD | | | |
| | Pub vs. | 3.365 | .001* | .01 |
| | Pub δ^{-/-} | | | |
| AP | Pre vs. Pub | .022 | 0.982 | .05 |
| Amplitude | Pub vs. | .0754 | 0.940 | .017 |
| | Pub-ZD | | | |
| | Pub vs. | .0456 | 0.964 | .017 |
| | Pub δ^{-/-} | | | |
| AP | Pre vs. Pub | .133 | 0.894 | .010 |
| half-width | Pre vs. | .0411 | 0.967 | .025 |
| | Pub-ZD | | | |
| | Pub vs. | 1.35 × 10−16 | 1.00 | .05 |
| | Pub δ^{-/-} | | | |

TABLE 13
ANOVA - Elevated plus maze

| Elevated plus | | | | |
|---|---|---|---|---|
| maze - FIG. 7 | Group DF | Res DF | F | P |
| stress effects | 11 | 50 | 7.77* | <.0001 |
| on open arm | | | | |
| time | | | | |
| Control | 8 | 54 | 0.432 | .897 |
| open arm | | | | |
| time | | | | |

TABLE 14
Least Significant Difference planned post-hoc comparisons*

| Group | Comparison | P |
|---|---|---|
| Pre-pubertal | Pubertal | .000148* |
| | 3β-THP - Pre-pubertal | .029* |
| | Pre-pubertal - δ^{-/-} | .045* |
| | Pubertal - THP | .0000206* |
| Pubertal | Pre-pubertal | .000148* |
| | 3β-THP - Pre-pubertal | .0119* |
| | 3β-THP - Pubertal | .0196* |
| | Adult | .0000526* |
| | Pubertal - finasteride | .042* |
| | Pre-pubertal - THP | .0000000675* |

*Similar results obtained with Duncan's test. These results were based on percentage changes induced by stress. Similar statistical results were obtained when absolute values of open arm time were compared across groups.

### REFERENCES

1. Buchanan, C. M., Eccles, J. S. & Becker, J. B. Are adolescents the victims of raging hormones: evidence for activational effects of hormones on moods and behavior at adolescence. Psychol. Bull. 111, 62-107 (1992).
2. Hayward, C. & Sanborn, K. Puberty and the emergence of gender differences in psychopathology. J. Adolescent Health 30S, 49-58 (2002).
3. Modesti, P. A. et al. Changes in blood pressure reactivity and 24-hour blood pressure profile occurring at puberty. Angiology 45, 443-450 (2006).
4. Rudolph, U. et al. Benzodiazepine actions mediated by specific E-aminobutyric acidA receptor subtypes. Nature 401, 796-800 (1999).
5. Majewska, M. D., Harrison, N. L., Schwartz, R. D., Barker, J. L. & Paul, S. M. Steroid hormone metabolites are barbiturate-like modulators of the GABA receptor. Science 232, 1004-1007 (1986).
6. Purdy, R. H., Morrow, A. L., Moore, P. H., Jr. & Paul, S. M. Stress-induced elevations of gamma-aminobutyric acid type A receptor-active steroids in the rat. Proc. Natl. Acad. Sci. 88, 4553-4557 (1991).
7. Freeman, E. W., Purdy, R. H., Coutifaris, C., Rickels, K. & Paul, S. M. Anxiolytic metabolites of progesterone: correlation with mood and performance measures following oral progesterone administration to healthy female volunteers. Neuroendo. 58, 478-484 (1993).
8. Bitran, D., Dugan, M., Renda, P., Ellis, R. & Foley, M. Anxiolytic effects of the neuroactive steroid pregnanolone (3alpha-OH-5beta-pregnan-20-one) after microinjection in the dorsal hippocampus and lateral septum. Brain Res. 850, 217-224 (1999).
9. Chang, Y., Wang, R., Barot, S. & Weiss, D. S. Stoichiometry of a recombinant GABA-A receptor. J. Neurosci. 16, 534-541 (1990).
10. Wohifarth, K. M., Bianchi, M. T. & Macdonald, R. L. Enhanced neurosteroid potentiation of ternary GABA(A) receptors containing the delta subunit. J. Neurosci. 22, 1541-1549 (2002).
11. Bianchi, M. T., Haas, K. F. & Macdonald, R. L. Alpha1 and alpha6 subunits specify distinct desensitization, deactivation and neurosteroid modulation of GABA(A) receptors containing the delta subunit. Neuropharm. 43, 492-502 (2002).
12. Belelli, D., Casula, A., Ling, A. & Lambert, J. J. The influence of subunit composition on the interaction of neurosteroids with GABA(A) receptors. Neuropharm. 43, 651-661 (2002).
13. Wei, W., Zhang, N., Peng, Z., Houser, C. R. & Mody, I. Perisynaptic localization of delta subunit-containing GABA(A) receptors and their activation by GABA spillover in the mouse dentate gyrus. J. Neurosci. 23, 10650-10661 (2003).
14. Stell, B. M., Brickley, S. G., Tang, C. Y., Farrant, M. & Mody, I. Neuroactive steroids reduce neuronal excitability by selectively enhancing tonic inhibition mediated by delta subunit-containing GABA-A receptors. Proc. Natl. Acad. Sci. 100, 14439-14444 (2003).
15. Corpechot, C. et al. Brain neurosteroids during the mouse oestrous cycle. Brain Res. 766, 276-280 (1997).
16. Wisden, W., Laurie, D. J., Monyer, H. & Seeburg, P. The distribution of 13 GABA-A receptor subunit mRNAs in the rat brain. I. Telencephalon, diencephalon, mesencephalon. J. Neurosci. 12, 1040-1062 (1992).
17. Smith, S. S. et al. GABA_{A} receptor □04 subunit suppression prevents withdrawal properties of an endogenous steroid. Nature 392, 926-929 (1998).
18. Smith, S. S., Ruderman, Y., Frye, C. A., Homanics, G. E. & Yuan, M. Steroid withdrawal in the mouse results in anxiogenic effects of 3α,5β-THP: A possible model of premenstrual dysphoric disorder. Psychopharm. 186, 323-333 (2006).
19. Sundstrom-Poromaa, I. et al. Hormonally regulated □4□2□ GABA_{A} receptors are a target for alcohol Nat. Neurosci. 5, 721-722 (2002).
20. Staley, K. J. & Mody, I. Shunting of excitatory input to dentate gyrus granule cells by a depolarizing GABA-A receptor-mediated postsynaptic conductance. J. Neurophysiol. 68, 197-212 (1992).
21. Staley, K. J. & Proctor, W. R. Modulation of mammalian dendritic GABA(A) receptor function by the kinetics of Cl- and HCO3-transport. J. Physiol. 519, 693-712 (1999).
22. Alger, B. E. & Nicoll, R. A. Pharmacological evidence for two kinds of GABA receptor on rat hippocampal pyramidal cells studied in vitro. J. Physiol. 328, 125-141 (1982).
23. Lambert, N. A., Borroni, A. M., Grover, L. M. & Teyler, T. J. Hyperpolarizing and depolarizing GABA-A receptor-mediated dendritic inhibition in area CA1 of the rat hippocampus. J. Neurophysiol. 66, 1538-1548 (1991).
24. Wu, Y., Wang, W. & Richerson, G. GABA transaminase inhibition induces spontaneous and enhances depolarization-evoked GABA efflux via reversal of the GABA transporter. J. Neurosci. 21, 2630-2639 (2001).
25. Miyazawa, A., Fujiyoshi, Y. & Unwin, N. Structure and gating mechanism of the acetylcholine receptor pore. Nature 424, 949-955 (2003).
26. Kelley, S. P., Dunlop, J. I., Kirkness, E., Lambert, J. J. & Peters, J. A. A cytoplasmic region determines single-channel conductance in 5-HT3 receptors. Nature 424, 321-324 (2003).
27. Chen, J., Mitcheson, J. S., Lin, M. & Sanguinetti, M. C. Functional roles of charged residues in the putative voltage sensor of the HCN2 pacemaker channel. J. Biol. Chem. 275, 36465-36471 (2000).
28. Fadalti, M. et al. Changes of serum allopregnanolone levels in the first 2 years of life and during pubertal development. Pediatr. Res. 46, 323-327 (1999).
29. Caraiscos, V. B. et al. Tonic inhibition in mouse hippocampal CA1 pyramidal neurons is mediated by alpha 5 subunit-containing gamma-aminobutyric acid type A receptors. Proc. Natl. Acad. Sci. 101, 3662-3667 (2004).
30. Stell, B. M. & Mody, I. Receptors with different affinities mediate phasic and tonic GABA(A) conductances in hippocampal neurons. J. Neurosci. 22, RC223 (2002).
31. Perkins, K. L. Cell-attached voltage-clamp and current-clamp recording and stimulation techniques in brain slices. J. Neurosci. Methods 154, 1-18 (2006).
32. Belelli, D., Peden, D. R., Rosahl, T. W., Wafford, K. & Lambert, J. J. Extrasynaptic GABA-A receptors for thalamocortical neurons: A molecular target for hypnotics. J. Neurosci. 25, 11513-11520 (2005).
33. Ulrich, D. & Huguenard, J. R. Nucleus-specific chloride homeostasis in rat thalamus. J. Neurosci. 17, 2348-2354 (1997).
34. Girdler, S. S., Beth Mechlin, M., Light, K. C. & Morrow, A. L. Ethnic differences in allopregnanolone concentrations in women during rest and following mental stress. Psychophysiology 43, 331-336 (2006).
35. McEwen, B. S. Stressed or stressed out: what is the difference? J. Psychiatr Neurosci. 30, 315-318 (2005).
36. Lundgren, P., Stromberg, J., Backstrom, T. & Wang, M. Allopregnanolone-stimulated GABA-mediated chloride ion flux is inhibited by 3beta-hydroxy-5alpha-pregnan-20-one (isoallopregnanolone). Brain Res. 982, 45-53 (2003).
37. Ramsey, I. S., Moran, M. M., Chong, J. A. & Clapham, D. E. A voltage-gated proton-selective channel lacking the pore domain. Nature 440, 1213-1216 (2006).
38. Olsen, R. W. & Snowman, A. Chloride-dependent enhancement by barbiturates of gamma-aminobutyric acid receptor binding. J. Neurosci. 2, 1812-1823 (1982).
39. Turner, J. H. & Raymond, J. R. Interaction of calmodulin with the serotoin 5-hydroxytryptamine-2A receptor. A putative regulator of G protein coupling and receptor phosphorylation by protein kinase C. J. Biol. Chem. 280, 30741-30750 (2005).
40. Haas, K. F. & Macdonald, R. L. GABA_{A} receptor subunit gamma2 and delta subtypes confer unique kinetic properties on recombinant GABA_{A} receptor currents in mouse fibroblasts. J. Physiol. 514, 27-45 (1999).
41. Chen, X. et al. Inhibition of a background potassium channel by Gq protein alpha-subunits. Proc. Natl. Acad. Sci. 103, 3422-3427 (2006).
42. Prescott, S. A. & de Koninck, Y. Gain control of firing rate by shunting inhibition: roles of synaptic noise and dendritic saturation. Proc. Natl. Acad. Sci. 100, 2076-2081 (2003).
43. Bai, D. et al. Distinct functional and pharmacological properties of tonic and quantal inhibitory postsynaptic currents mediated by γ-aminobutyric acid(A) receptors in hippocampal neurons. Molec. Pharmacol. 59, 814-824 (2000).
44. Maguire, J. L., Stell, B. M., Rafizadeh, M. & Mody, I. Ovarian cycle-linked changes in GABA(A) receptors mediating tonic inhibition alter seizure susceptibility and anxiety. Nat. Neurosci. 8, 797-804 (2005).
45. Romeo, R. D. Neuroendocrine and behavioral development during puberty: a tale of two axes. Vitam. Horm. 71, 1-25 (2005).
46. Barbaccia, M. L., Serra, M., Purdy, R. H. & Biggio, G. Stress and neuroactive steroids. Int. Rev. Neurobiol. 46, 243-272 (2001).
47. Mele, P. et al. Increased expression of the gene for the Y1 receptor of the neuropeptide Y in the amygdala and paraventricular nucleus of Y1 R/LacZ transgenic mice in response to restraint stress. J. Neurochem. 89, 1471-1478 (2004).
48. Freeman, E. W., Frye, C. A., Rickels, K., Martin, P. A. & Smith, S. S. Allopregnanolone levels and symptom improvement in severe premenstrual syndrome. J. Clin. Psychopharmacol. 22, 516-520 (2002).
49. Schmidt, P., Nieman, L., Danaceau, M., Adams, L. & Rubinow, D. Differential behavioral effects of gonadal steroids in women with premenstrual syndrome. New England J. Med. 338, 209-216 (1998).
50. Andreen, L. et al. Relationship between allopregnanolone and negative mood in postmenopausal women taking sequential hormone replacement therapy with vaginal progesterone. Psychoneuroendo. 30, 212-224 (2004).
51. Kawai, J., et al. 2001. Functional annotation of a full-length mouse cDNA collection. Nature. 409: 685-690
52. Mu, W. Cheng, Q., Yang, J., and D. R. Burt. 2002. Alternative Splicing of the GABA(A) RMeceptor Alpha 4 Subunit Creates a Severely Truncated mRNA. Brain Res. Bull. 58:447-484.

### SUPPLEMENTARY REFERENCE LIST

1. Mihalek, R. M. et al. GABA(A)-receptor delta subunit knockout mice have multiple defects in behavioral responses to ethanol. Alcohol Clin Exp Res 25, 1708-1718 (2001).
2. Frye, C. A. & Vongher, J. M. Progesterone and 3alpha,5alpha-THP enhance sexual receptivity in mice. Behav. Neurosci. 115, 1118-1128 (2001).
3. Corpechot, C. et al., Brain neurosteroids during the mouse oestrous cycle. Brain Res 766, 276-280 (1997).
4. Frye, C. A., Bayon, L. E., Pursnani, N. K. & Purdy, R. H. The neurosteroids, progesterone and 3alpha,5alpha-THP, enhance sexual motivation, receptivity, and proceptivity in female rats. Brain Res 808, 72-83 (1998).
5. Purdy, R. H., Morrow, A. L., Moore, P. H., Jr. & Paul, S. M. Stress-induced elevations of gamma-aminobutyric acid type A receptor-active steroids in the rat. PNAS 88, 4553-4557 (1991).
6. Rodbard, D. & Hutt, D. M. Statistical analysis of radioimmunoassay and immunoradiometric assays: a generalized, weighted iterative, least squares method for logistic curve fitting. Int. Atomic Energy Agency (ed) 209-223 (1974).
7. Smith, S. S. et al. GABA_{A} receptor α4 subunit suppression prevents withdrawal properties of an endogenous steroid. Nature 392, 926-929 (1998).
8. Aoki, C., Rogrigues, S. & Kurose, H. Use of electron microscopy in the detection of adrenergic receptors. Machida, C. A. Methods in Molecular Biology: Adrenergic Receptor Protocols. (126), 535-563. 2000. New York, Humana Press.
9. Fujisawa, S., Shirao, T. & Aoki, C. In vivo, competitive blockade of N-methyl-D-aspartate receptors induces rapid changes in filamentous actin and drebrin A distributions within dendritic spines of adult rat cortex. Neuroscience 140, 1177-1187 (2006).
10. Lovick, T. A., Griffiths, J. L., Dunn, S. M. & Martin, I. L. Changes in GABA(A) receptor subunit expression in the midbrain during the oestrous cycle in Wistar rats. Neuroscience 131, 397-405 (2005).
11. Peng, Z., Huang, C. S., Stell, B. M., Mody, I. & Houser, C. R. Altered expression of the delta subunit of the GABA-A receptor in a mouse model of temporal lobe epilepsy. J. Neurosci 24, 8629-8639 (2004).
12. Boileau, A. J., Baur, R., Sharkey, L. M., Sigel, E. & Czajkowski, C. The relative amount of cRNA coding for gamma2 subunits affects stimulation by benzodiazepines in GABA(A) receptors expressed in Xenopous oocytes. Neuropharm. 43, 695-700 (2002).
13. Sundstrom-Poromaa, I. et al., Hormonally regulated α4 β2δ GABA_{A} receptors are a target for alcohol. Nat Neurosci 5, 721-722 (2002).
14. Smith, S. S. & Gong, Q. H. Neurosteroid administration and withdrawal alter GABA-A receptor kinetics in CA1 hippocampus of female rats. J Physiol 564, 421-436 (2005).
15. Bianchi, M. T. & Macdonald, R. L. Neurosteroids shift partial agonist activation of GABA(A) receptor channels from low- to high-efficacy gating patterns. J. Neurosci. 23, 10934-10943 (2003).
16. Brown, N., Kerby, J., Bonnert, T. P., Whiting, P. J. & Wafford, K. A. Pharmacological characterization of a novel cell line expressing human alpha(4)beta(3)delta GABA(A) receptors. Br. J. Pharmacol 136, 965-974 (2002).
17. Saxena, N. C., Neelands, T. R. & Macdonald, R. L. Contrasting actions of lanthanum on different recombinant gamma-aminobutyric acid receptor isoforms expressed in L929 fibroblasts. Mol Pharmacol 51, 328-335 (1997).
18. Feng, H.-J. et al. Delta subunit susceptibility variants E177A and R220H associated with complex epilepsy alter channel gating and surface expression of alpha4-beta2-delta GABA-A receptors. J. Neurosci. 26, 1499-1506 (2006).
19. Benson, J. A., Low, K., Keist, R., Mohler, H. & Rudolph, U. Pharmacology of recombinant gamma-aminobutyric acid-A receptors rendered diazepam-insensitive by point-mutated alpha-subunits. FEBS Letter 431, 400-404 (1998).
20. Hsu F.-C., Waldeck, R., Faber, D. S. & Smith SS. Neurosteroid effects on GABAergic synaptic plasticity in hippocampus. J. Neurophysiol. 89, 1929-1940 (2003).
21. Williams, J. R. & Payne, J. A. Cation transport by the neuronal K(+)-Cl(−) cotransporter KCC2: thermodynamics and kinetics of alternate transport modes. Am. J. Physiol. Cell Physiol. 287, C919-C931 (2004).
22. Stell, B. M., Brickley, S. G., Tang, C. Y., Farrant, M. & Mody, I. Neuroactive steroids reduce neuronal excitability by selectively enhancing tonic inhibition mediated by delta subunit-containing GABA-A receptors. Proc Natl Acad Sci 100, 14439-14444 (2003).
23. Caraiscos, V. B. et al. Tonic inhibition in mouse hippocampal CA1 pyramidal neurons is mediated by alpha 5 subunit-containing gamma-aminobutyric acid type A receptors. Proc Natl Acad Sci 101, 3662-3667 (2004).
24. Shen, H., Gong, Q. H., Yuan, M. & Smith, S. S. Short-term steroid treatment increases delta GABA-A receptor subunit expression in rat CA1 hippocampus: pharmacological and behavioral effects. Neuropharmacology 49, 573-586 (2005).
25. Mason, M. J., Simpson, A. K., Mahaut-Smith, M. P. & Robinson, H. P. The interpretation of current-clamp recordings in the cell-attached patch-clamp configuration. Biophys J 88, 739-750 (2005).
26. Perkins K L. Cell-attached voltage-clamp and current-clamp recording and stimulation techniques in brain slices. J Neurosci Methods 154, 1-18 (2006).
27. Rosenkranz, J. A. & Johnston, D. Dopaminergic regulation of neuronal excitability through modulation of 1 h in layer V entorhinal cortex. J. Neurosci. 26, 3229-3244 (2006).
28. Kaczorowski, C. C., Disterhoft, J. F. & Spruston, N. Stability and plasticity of intrinsic membrane properties in hippocampal CA1 pyramidal neurons: effects of internal anions. J. Physiol. 578, 799-818 (2007).
29. Bennett, B. D. & Wilson, C. J. Spontaneous activity of neostriatal cholinergic interneurons in vitro. J Neurosci 19, 5586-5596 (1999).
30. Ulrich, D. & Huguenard, J. R. Nucleus-specific chloride homeostasis in rat thalamus. J. Neurosci. 17, 2348-2354 (1997).
31. Pellow, S., Chopin, P., File, S. E. & Briley, M. Validation of open:closed arm entries in an elevated plus maze as a measure of anxiety in the rat. 489. J. Neurosci. Methods 14, 149-167 (1995).
32. Smith, S. S., Ruderman, Y., Frye, C. A., Homanics, G. E. & Yuan M. Steroid withdrawal in the mouse results in anxiogenic effects of 3α,5β3-THP: A possible model of premenstrual dysphoric disorder. Psychopharmacology 186, 323-333 (2006).
33. Mele, P. et al. Increased expression of the gene for the Y1 receptor of the neuropeptide Y in the amygdala and paraventricular nucleus of Y1R/LacZ transgenic mice in response to restraint stress. J Neurochem 89, 1471-1478 (2004).
34. Baoglu, C., Cetin, M., Semiz, U. B., Agargun, M. Y. & Ebrinc, S. Premenstrual exacerbation and suicidal behavior in patients with panic disorder. Compr. Psychiatry 41, 103-105 (2000).
35. Higashi T, Takido N & Shimada K. Studies on neurosteroids XVII. Analysis of stress-induced changes in neurosteroid levels in rat brains using liquid chromatography-electron capture atmospheric pressure chemical ionization-mass spectrometry. Steroids 70, 1-11 (2005).
36. Zar, J. Biostatistical Analysis. Prentice-Hall, Inc., Upper Saddle River, N.J. (1999).
37. Wisden, W., Laurie, D. J., Monyer, H. & Seeburg, P. The distribution of 13 GABA-A receptor subunit mRNAs in the rat brain. I. Telencephalon, diencephalon, mesencephalon. J Neurosci 12, 1040-1062 (1992).

What is claimed is:

1. A method for treating anxiety or irritability in a subject, said method comprising: administering to the subject an effective amount of an antagonist of allopregnanolone (THP).

2. The method of claim 1 wherein the subject is undergoing a stage selected from the group consisting of: entering or having reached puberty, suffering from pre-menstrual syndrome (PMS), entering or having reached post-partem stage, entering or having reached menopause, and suffering from chronic stress.

3. A method for treating anxiety or irritability in a subject, said method comprising administering to the subject an effective amount of a regulator which decreases expression of the alpha 4 subunit of GABA.

4. The method of claim 3 wherein the subject is undergoing a stage selected from the group consisting of: entering or having reached puberty, suffering from pre-menstrual syndrome (PMS), entering or having reached post-partem stage, entering or having reached menopause, and suffering from chronic stress.

5. The method of claim 3 wherein the regulator is gabadoxbol (THIP).

6. An isolated mutant alpha 4 subunit of GABA_{A} receptor protein wherein the mutant protein has a neutral or non-basic amino acid residue substituted for the arginine residue at position 353 of the wild type mature protein.

7. An isolated nucleic acid molecule encoding the mutated protein of claim 6.

8. A method of treating anxiety or irritability in a subject, said method comprising administering to the subject an effective amount of a vector comprising the isolated nucleic acid molecule of claim 7 operably linked to a promoter which functions in the human brain.

9. The method of claim 8 wherein the subject is undergoing a stage selected from the group consisting of: entering or having reached puberty, suffering from pre-menstrual syndrome (PMS), entering or having reached post-partem stage, entering or having reached menopause, and suffering from chronic stress.

10. The method of claim 8 wherein the promoter is selected from the group consisting of CAM-kinase II, gamma-8 membrane-associated guanylate kinase and KCC2 (K-Cl co-transporter).

11. A method for identifying an antagonist of THP, said method comprising: (a) expressing α4 β2δ GABA_{A} receptors in eukaryotic cells; (b) applying a drug to the eukaryotic cells of (a); (c) measuring GABA_{A} gated currents at α4 β2δ GABA_{A} receptors in the treated cells of (b); and (d) correlating a increase in outward currents recorded at α4 β2δ GABA_{A} receptors when compared to a eukaryotic cell population having THP application, with the identification of an antagonist of THP.

12. A vector comprising the isolated nucleic acid molecule of claim 7 operably liked to a promoter which functions in prokaryotic cells.

13. A vector comprising the isolated nucleic acid molecule of claim 8 operably liked to a promoter which functions in eukaryotic cells.

14. A host cell comprising the vector of claim 13 or 14.
